(19) United States
(12) Patent Application Publication
(10) Pub. No.: US 2022/0047469 A1
(43) Pub. Date: Feb. 17, 2022

(54) THERMORESPONSIVE OIL-IN-WATER NANOEMULSION

(71) Applicants: L'OREAL, Paris (FR); MASSACHUSETTS INSTITUTE OF TECHNOLOGY, Cambridge, MA (US)

(72) Inventors: Brady ZARKET, Union, NJ (US); Patrick S. DOYLE, Cambridge, NJ (US); Abu Zayed Md BADRUDDOZA, Clark, NJ (US); Seyedmeysam HASHEMNEJAD, Clark, NJ (US); Samiul AMIN, Clark, NJ (US); Guillaume CASSIN, Paris (FR); Carlos Ricardo CASTANEDA, San Antonio, TX (US)

(21) Appl. No.: 17/297,736

(22) PCT Filed: Nov. 25, 2019
(86) PCT No.: PCT/US2019/062929

§ 371 Date: May 27, 2021

Related U.S. Application Data
(60) Provisional application No. 62/773,606, filed on Nov. 30, 2018.

(51) Int. Cl. A61K 8/04; A61K 9/06; A61K 47/10; A61K 47/14; A61K 47/26; A61K 8/86; A61K 8/37; A61K 8/60; A61Q 19/00; A61Q 5/00

(52) U.S. Cl. CPC A61K 8/042; A61K 9/06; A61K 47/10; A61K 47/14; A61K 47/26; A61K 2800/24; A61K 8/37; A61K 8/608; A61Q 19/00; A61Q 5/00; A61K 2800/21; A61K 8/86

(57) ABSTRACT

The present disclosure relates to thermoresponsive oil-in-water nanoemulsions that include: (a) one or more amphiphilic triblock copolymers; (b) one or more oils in the form of nanoemulsion droplets having an average size of about 20 nm to about 500 nm; (c) about 5 to about 40 wt. % of one or more surfactants; (d) one or more nonionic co¬ surfactants; and (e) water, wherein the nonemulsion undergoes a sol-to-gel transition at about 20° C. to about 50° C. One or more lipophilic active ingredients and/or one or more hydrophilic active ingredients may be incorporated into the nanoemulsions. The nanoemulsions are particularly useful for medical, pharmaceutical, and cosmetic applications.

### FIELD OF THE DISCLOSURE

The present disclosure relates to thermoresponsive oil-in-water nanoemulsions that are particularly useful for medical, pharmaceutical, and cosmetic applications.

### BACKGROUND

Hydrogels are a unique class of materials with tuneable physicochemical and biological properties that are well suited for a variety of biomedical applications. Their crosslinked three-dimensional (3D) network structures contain a significant amount of water, yet still behave like an elastic solid. Hydrogels are typically synthesized by chemical (covalent) or physical (noncovalent) crosslinking of natural or synthetic polymers and other small molecules.

Physically crosslinked hydrogels, which avoid the use of toxic chemical crosslinking reagents, are of great interest due to their inherent reversibility and the dynamics of their physical interactions. The dynamic nature of a physically self-assembled gel network can help prevent permanent dissociation of the microstructure and promotes self-healing. The self-healing properties of hydrogels have been shown in different physical crosslinking strategies such as hydrophobic interactions, hydrogen bonding, electrostatic interactions, and host-guest interactions. The physically self-assembled hydrogels can also be formed in response to an external stimuli such as solvent properties, pH, redox, light, and temperature. Among these stimuli-responsive systems, thermal-responsive hydrogels that undergo a sol-to-gel transition at ambient to physiological temperature are highly desirable for biological applications.

Most of the thermogelling polymer hydrogels reported in the literature are based on synthetic polymers with temperature-sensitive moieties. Ideally, hydrogels will possess certain mechanical properties for use as an injectable vehicle or in topical formulations. For example, substantial decreases in viscosity as they flow (shear-thinning), rapidly forming a gel at elevated temperature, and fast self-healing properties.

Organohydrogels containing hydrophilic and lipophilic domains have gained great interest in recent years. Emulsion-based organohydrogels are highly desirable as they allow for the facile loading of bioactive compounds. The encapsulation procedures require only mild dissolution processes, which eliminate any additional chemical modification of bioactive components. The immiscible domains (e.g. oil and water) can be achieved through emulsification methods, which are broadly classified as either high- or low-energy processes. Nanoscale emulsions with sizes on the order of 100 nm (so-called nanoemulsions) provide an efficient and facile approach to encapsulating both polar and non-polar functional biomolecules for co-delivery of dissimilar therapeutics. Multiple strategies can be employed to obtain thermogelling emulsion-based organohydrogels which are mostly based on entrapping the dispersed phase via thermally gelling the continuous phase.

### SUMMARY OF THE DISCLOSURE

The present disclosure relates to thermoresponsive oil-in-water nanoemulsions. The nanoemulsions spontaneously form a gel at elevated temperatures, display shear-thinning behavior in both liquid and gel states, and rapidly self-recover following the cessation of applied stress. Without wishing to be bound by any particular theory, it is believed that hydrophobic groups in the central region of the amphiphilic triblock copolymers interact with nanoemulsion droplets, thereby functioning as “gelators.” The nanoemulsion can be prepared at ambient temperatures, allowing for easy manufacture and scale-up. Furthermore, the ingredients of the nanoemulsions are safe (non-toxic) and biocompatible. In view of their biocompatibility and unique physical properties, the nanoemulsions are useful in medical, pharmaceutical, and cosmetic applications.

The thermoresponsive oil-in-water nanoemulsions typically include:
(a) one or more amphiphilic triblock copolymers;
(b) one or more oils in the form of nanoemulsion droplets having an average size (average diameter) of about 20 nm to about 500 nm;
(c) about 5 to about 40 wt. % of one or more surfactants;
(d) one or more nonionic co-surfactants; and
(e) water.

The nanoemulsions typically undergo a sol-to-gel transition at physiological temperatures, for example, at a temperature of about 20° C. to about 50° C.

The nanoemulsions may optionally include one or more lipophilic active ingredients and/or one or more hydrophilic active ingredients. The one or more lipophilic active ingredients can be solubilized in the one or more oils (i.e., in the nanoemulsion droplets) while the one or more hydrophilic active agents can be solubilized in the aqueous phase of the nanoemulsions.

Also described are methods for making the thermoresponsive oil-in-water nanoemulsions. For instance, the nanoemulsions can be prepared by:
(i) combining one or more oils, one or more surfactants, and optionally one or more nonionic co-surfactants;
(ii) adding water to the combination;
(iii) forming a nanoemulsion and optionally adding one or more nonionic co-surfactants; and
(iv) adding one or more amphiphilic triblock copolymers to the nanoemulsion.

The method may be carried out, for example, at a temperature of about 0° C. to about 40° C.

Finally, the present disclosure relates to therapeutic and non-therapeutic methods of using the nanoemulsions, for example, in medical, pharmaceutical, and/or cosmetic applications.

### BRIEF DESCRIPTION OF THE DRAWINGS

Implementation of the present technology will now be described, by way of example only, with reference to the attached figures, wherein:

FIG. 1 is a schematic diagram showing the hypothesized gelation mechanism, wherein: (a) illustrates formation of the oil-in-water nanoemulsion using a low energy process at room temperature (RT); (b) illustrates the addition of an amphiphilic triblock polymer (ATC) to provide thermoresponsive behavior; and (c) illustrates gel formation;

FIG. 2 illustrates the rheological behavior of a the thermoresponsive oil-in-water nanoemulsions, wherein: (a) shows the evolution of the dynamic moduli in a small amplitude oscillatory shear temperature ramp experiment from 10° C. to 50° C. with a ramp rate of 2° C./min; and (b) shows the linear viscoelastic spectra at different temperatures.

FIG. 3 provides differential scanning calorimetry (DSC) traces of: (a) aqueous solutions of different amphiphilic triblock copolymer concentrations; and (b) the aqueous solution (1.2%), the subnatant solution (continuous phase), and nanoemulsion;

FIG. 4 shows the effect of nanoemulsion droplet size on rheological properties and gelation temperature T_{gel} with shear moduli as a function of temperature and frequency at 50° C. FIG. 4(a)-(c) show the evolution of the dynamic moduli in a small amplitude oscillatory shear temperature ramp experiment for droplets of sizes of: (a) 53 nm; (b) 72 nm; and (c) 115 nm. FIG. 4(d)-(f) show the linear viscoelastic spectra for the same nanoemulsions.

FIG. 5 shows the effect of a nonionic co-surfactant (PEG 400) on rheological properties and gelation temperature T_{gel} with shear moduli as a function of temperature and frequency at 50° C., wherein nanoemulsion droplet size (53 nm) and volume fraction (ϕ=0.24) are kept constant for all formulations. The only difference in the chemical composition of the samples is the amount of added co-surfactant. FIG. 5(a)-(c) show the evolution of the dynamic moduli in a small amplitude oscillatory shear temperature ramp experiment at co-surfactant concentrations of: (a) 5 wt. %, (b) 7.5 wt. %, and (c) 10 wt. %. FIG. 5(d)-(f) show the linear viscoelastic spectra for the same systems;

FIG. 6 shows the linear and nonlinear rheological characterization of the nanoemulsions below (20° C.) and above (50° C.) the gel point, wherein: (a) shows a steady shear rheology at 20° C.; (b) shows the viscoelastic moduli in a temperature jump from sol state (20° C.) to gel state (50° C.); (c) shows an optical image of mushroom-shaped gel particles formed by dripping a room temperature thermoresponsive nanoemulsion (using a 15-gauge needle) into a hot water bath at 50° C.; (d) shows shear stress; (e) and (f) show changes in the moduli indicating breaking and recovery of the structure under applied stress of 500 Pa and 5 Pa, respectively; and (g) shows a cyclic flow sweep experiment from low to high shear rate (up) and from high to low shear rate (down) at 50° C.

It should be understood that the various aspects are not limited to the arrangements and instrumentality shown in the drawings.

### DETAILED DESCRIPTION OF THE DISCLOSURE

The present disclosure relates to thermoresponsive oil-in-water nanoemulsions comprising:
a. about 1 to about 15 wt. % of one or more amphiphilic triblock copolymers;
b. about 10 to about 50 wt. % of one or more oils in the form of nanoemulsion droplets having an average size of about 20 nm to about 500 nm;
c. about 1 to about 25 wt. % of one or more surfactants;
d. about 0.1 to about 10 wt. % of one or more nonionic co-surfactants; and
e. about 25 to about 75 wt. % of water;

wherein the nonemulsion undergoes a sol-to-gel transition at a temperature of about 20° C. to about 50° C.

In some instances, the sol-to-gel transition may occur at a temperature of about 25° C. to about 50° C., about 30° C. to about 50° C., about 35° C. to about 50° C., about 40° C. to about 50° C., about 20° C. to about 45° C., about 25° C. to about 45° C., about 30° C. to about 45° C., about 35° C. to about 45° C., about 20° C. to about 40° C., about 25° C. to about 40° C., about 30° C. to about 40° C., about 20° C. to about 35° C., or about 25° C. to about 35° C.

The nanoemulsions typically exhibit shear-thinning behavior in both liquid and gel states, and rapidly self-recover following the cessation of applied stress. Shear thinning is the non-Newtonian behavior of fluids whose viscosity decreases under shear strain. Classical “Newtonian” fluids have a viscosity which is essentially independent of shear rate. “Non-Newtonian fluids,” however, demonstrate a viscosity which either decreases or increases with increasing shear rate, e.g., the fluids are “shear thinning” or “shear thickening”, respectively. Accordingly, the nanoemulsions of the instant case may be referred to as “viscoelastic non-Newtonian nanoemulsions having shear thinning properties.” Non-Newtonian fluids, especially of multi-phase nature (e.g., nanoemulsions) do not conform to the Newtonian postulate of the linear relationship between shear stress and shear rate in simple shear. Likewise, the apparent viscosity, defined as shear stress/shear rate, is not constant and is a function of shear stress or shear rate.

In some instances, the nanoemulsions of the instant case are translucent or transparent. The term “translucent” as used herein with respect to a translucent nanoemulsion means that the nanoemulsion permits the passage of light but does not necessarily allow for detailed objects to be distinguished. The term “transparent” with respect to a transparent nanoemulsion, however, means that the nanoemulsion permits the passage of light and also makes possible the distinguishing of objects. In other words, a transparent nanoemulsion is clearer than a translucent nanoemulsion. Colorants can be included in a translucent or transparent nanoemulsion without destroying the translucent or transparent characteristics of the composition. In other words, the terms do not necessarily require compositions to be color-free like pure water, but includes colored products that appear, for example, like colored glass. The term “transparent” with respect to nanoemulsion of the instant disclosure indicates that the nanoemulsion has transmittance of at least 80% at a wavelength of 600 nm, for example measured using a Lambda 40 UV-visible spectrometer, at a concentration of 0.5% by weight in water. The nanoemulsion may have, for example, a transmittance of at least 80%, at least 90%, or at least 95% at a wavelength of 600 nm, measured, for example, using a Lambda 40 UV-visible spectrometer. The term “clear” is interchangeable with the term “transparent” for purposes of the instant disclosure.

In some cases, the nanoemulsions are free or essentially free of silicones. For example, the nanoemulsions may include less than about 3 wt. %, 2 wt. %, 1 wt. %, or 0.5 wt. % of silicones (or preferably no silicones). Non-limiting examples of silicones include amine-functionalized silicones (e.g., amodimethicone), dimethicone, bis-aminopropyl dimethicone, trimethyl silylamodimethicon, etc.

Amphiphilic Triblock Copolymers

The term, “amphiphilic triblock copolymer” refers to a copolymer possessing both hydrophilic and lipophilic properties. For example, the amphiphilic triblock copolymers typically include at least one hydrophilic block and at least on hydrophobic block. In some instances, the amphiphilic triblock copolymers preferably include a lipophilic central block flanked by hydrophilic outer blocks. In some instances, the amphiphilic triblock copolymers are water-soluble triblock copolymers composed of polyethylene oxide (PEO), and polypropylene oxide (PPO) denoted as PEO—PPO-PEO or (EO)ₙ₁(PO)m(EO)ₙ₂ or HO(C₂H₄O)ₐ(C₃H₆O)_{b}(C₂H₄O)_{c}H (see, e.g., Schmolka, Supra; Alexandridis & Hatton, Colloids and Surfaces 96:1-46, (1995), which is incorporated herein by reference in its entirety). It is preferable that a and c are independently from 1-150 units and b ranges from 10-200 units with the overall molecular weight ranging from 1,000 to 50,000 daltons. Particularly preferred are those where a equals c, and b ranges from 10-200 units.

Others examples of amphiphilic triblock copolymers include those where the central block is composed of other amphiphilic, charged or uncharged monomeric groups that are capable of interacting with the oil droplets of the nanoemulsion (see, e.g., Kataoka et al. J. Controlled Release 24:119-132, (1993), which is incorporated herein by reference in its entirety). These moieties may be selected depending on the properties (polarity, charge, aromatic character, etc.).

In some cases, the amphiphilic triblock copolymers are symmetric and/or non-symmetric triblock copolymers and dendrimer types. Symmetrical triblock copolymers are particularly useful, for example, those composed of PEO—PPO-PEO, where the hydrophobic PPO provides methyl groups that are believed to interact with the oil droplets. PEO confers water solubility to the copolymer, although the hydrogen bonding interactions of the ether oxygen with water molecules probably occurs along the entire copolymer. These copolymers are available from a number of commercial sources such as BASF Corporation (PLURONICS.series) and ICI (SYNPERONIC series). In the numeric naming system for both the series, the last digit of the copolymer number multiplied by 10 gives the approximate percent molecular weight of the hydrophilic blocks (PEO).

The amphiphilic triblock copolymers are preferably selected from symmetric A-B-A and non-symmetric A-B-A′ type triblock copolymers. For example, the amphiphilic triblock copolymers are preferably a polyoxyethylene polyoxypropylene block copolymer of the formula HO(C₂H₄O)ₐ(C₃H₆O)_{b}(C₂H₄O)_{c}H, where a and c are independently 1-150 units and b is 10-200 units, with the overall molecular weight ranging from 1,000 to 50,000 daltons. Accordingly, the amphiphilic triblock copolymer may be selected fro poloxamers, wherein a-c-1 to 150 and b-10-200 unites. Non-limiting examples include poloxamer 403 (P123), poloxamer 407 (F127), poloxamer 402 (L122), poloxamer 181 (L61), poloxamer 401 (L121), poloxamer 185 (P65), poloxamer 188 (F68) and poloxamer 338 (F108). In some instances, a combination of poloxamer 407 (F127) and poloxamer 188 (F68) are preferred.

The total amount of the one or more amphiphilic triblock copolymers in the nanoemulsions can vary but is typically about 1 to about 15 wt. %, based on the total weight of the nanoemulsion. In some instances, the total amount of the one or more amphiphilic triblock copolymers is about 1 to about 10 wt. %, about 1 to about 8 wt. %, about to about 6 wt. %, about 1 to about 5 wt. %, about 2 to about 15 wt. %, about 2 to about 10 wt. %, about 2 to about 8 wt. %, about 2 to about 6 wt. %, or about 2 to about 5 wt. %, based on the total weight of the nanoemulsion.

Oils

The oil typically has a melting temperature of less than 45° C. and a solubility in water of no greater than 1 part in 99 parts of water. Examples of oils include: hydrocarbon-based oils of plant origin, such as perhydrosqualene, liquid triglycerides of fatty acids comprising from 4 to 10 carbon atoms, such as heptanoic or octanoic acid triglycerides, or alternatively, for example, sunflower oil, maize oil, soybean oil, marrow oil, grapeseed oil, sesame seed oil, hazelnut oil, apricot kernel oil, macadamia oil, arara oil, coriander oil, castor oil, avocado oil, caprylic/capric acid triglycerides, such as those sold by the company Stearineries Dubois or those sold under the names MIGLYOL 810, 812 and 818 by the company Dynamit Nobel, jojoba oil and shea butter oil; synthetic esters and ethers, especially of fatty acids and/or of fatty alcohols, for instance the oils of formulae R¹COOR² and R¹OR² in which R¹ represents a fatty acid residue containing from 7 to 29 carbon atoms and R² represents a branched or unbranched hydrocarbon-based chain containing from 3 to 30 carbon atoms, for instance purcellin oil, isononyl isononanoate, isopropyl myristate, isopropyl palmitate, 2-ethylhexyl palmitate, 2-octyldodecyl stearate, isocetyl stearate, 2-octyldodecyl erucate or isostearyl isostearate; hydroxylated esters, for instance isostearyl lactate, octyl hydroxystearate, octyldodecyl hydroxystearate, diisostearyl malate, triisocetyl citrate, and fatty alkyl heptanoates, octanoates and decanoates; polyol esters, for instance propylene glycol dioctanoate, neopentyl glycol diheptanoate and diethylene glycol diisononanoate; and pentaerythritol esters, for instance pentaerythrityl tetraisostearate; volatile or non-volatile, linear or branched hydrocarbons, of mineral or synthetic origin, and derivatives thereof, other than the branched alkanes comprising from 8 to 18 carbon atoms, such as liquid petroleum jelly and hydrogenated polyisobutene such as PARLEAM oil; volatile linear alkanes comprising from 7 to 17 carbon atoms such as undecane or tridecane; fatty alcohols that are liquid at room temperature, containing from 8 to 26 carbon atoms, for instance octyldodecanol, 2-butyloctanol, 2-hexyldecanol, 2-undecylpentadecanol or oleyl alcohol.

As non-limiting examples of linear alkanes, mention may be made of n-heptane (C7), n-octane (C8), n-nonane (C9), n-decane (C10), n-undecane (C11), n-dodecane (C12), n-tridecane (C13) and n-tetradecane (C14), and mixtures thereof. According to a particular embodiment, the volatile linear alkane is chosen from n-nonane, n-undecane, n-dodecane, n-tridecane and n-tetradecane, and mixtures thereof.

Non-limiting examples of liquid triglycerides and oils of plant origin include alexandria laurel tree oil, avocado oil, apricot stone oil, barley oil, borage seed oil, calendula oil, canelle nut tree oil, canola oil, caprylic/capric triglyceride castor oil, coconut oil, corn oil, cotton oil, cottonseed oil, evening primrose oil, flaxseed oil, groundnut oil, hazelnut oil, glycereth triacetate, glycerol triheptanoate, glyceryl trioctanoate, glyceryl triundecanoate, hempseed oil, jojoba oil, lucerne oil, maize germ oil, marrow oil, millet oil, neopentylglycol dicaprylate/dicaprate, olive oil, palm oil, passionflower oil, pentaerythrityl tetrastearate, poppy oil, propylene glycol ricinoleate, rapeseed oil, rye oil, safflower oil, sesame oil, shea butter, soya oil, soybean oil, sweet almond oil, sunflower oil, sysymbrium oil, syzigium aromaticum oil, tea tree oil, walnut oil, wheat germ glycerides and wheat germ oil.

The use of natural oils can be particularly preferred, for instance, natural oils of plant origin, such as amaranth seed oil, apricot kernel oil, argan oil, avocado oil, babassu oil, cottonseed oil, borage seed oil, camelina oil, thistle oil, peanut oil, pomegranate seed oil, grapefruit seed oil, hemp oil, hazelnut oil, elderberry seed oil, currant seed oil, jojoba oil, linseed oil, macadamia nut oil, corn oil, almond oil, marula oil, evening primrose oil, olive oil, palm oil, palm kernel oil, Brazil nut oil, pecan nut oil, peach kernel oil, rapeseed oil, castor oil, sea buckthorn pulp oil, sea buckthorn kernel oil, sesame oil, soy bean oil, sunflower oil, grape seed oil, walnut oil, wild rose oil, wheat germ oil, and the liquid components of coconut oil, and the like. In one embodiment, the oil is a plant oil selected from palm oil, soybean oil, olive oil, coconut oil, and a mixture thereof.

In some instances, the oil is preferably an ester oil. Ester oils include, but are not limited to, fatty esters having at least 10 carbon atoms. These fatty esters include esters derived from fatty acids or alcohols (e.g., mono-esters, polyhydric alcohol esters, and di- and tri-carboxylic acid esters). The fatty esters hereof may include or have covalently bonded thereto other compatible functionalities, such as amides and alkoxy moieties (e.g., ethoxy or ether linkages, etc.)

The ester oil may be selected from: monoesters comprising at least 18 carbon atoms and even more particularly containing between 18 and 40 carbon atoms in total, in particular monoesters of formula R₁COOR₂ in which R₁ represents a linear or branched, saturated or unsaturated or aromatic fatty acid residue comprising from 4 to 40 carbon atoms and R₂ represents a hydrocarbon-based chain that is in particular branched, containing from 4 to 40 carbon atoms, on condition that the sum of the carbon atoms of the radicals R₁ and R₂ is greater than or equal to 18, for instance Purcellin oil (cetostearyl octanoate), isononyl isononanoate, C₁₂ to C₁₅ alkyl benzoate, 2-ethylhexyl palmitate, octyldodecyl neopentanoate, 2-octyldodecyl stearate, 2-octyldodecyl erucate, isostearyl isostearate, C₁₂-C₁₅ alkyl benzoates such as 2-octyldodecyl benzoate, alcohol or polyalcohol octanoates, decanoates or ricinoleates, isopropyl myristate, isopropyl palmitate, butyl stearate, hexyl laurate, 2-ethylhexyl palmitate, 2-hexyldecyl laurate, 2-octyldecyl palmitate or 2-octyldodecyl myristate.

Preferably, the ester oils are esters of formula R₁COOR₂ in which R₁ represents a linear or branched fatty acid residue comprising from 4 to 40 carbon atoms and R₂ represents a hydrocarbon-based chain that is in particular branched, containing from 4 to 40 carbon atoms, R₁ and R₂ being such that the sum of the carbon atoms of the radicals R₁ and R₂ is greater than or equal to 18.

In some instances, the ester oil may be selected from diisobutyl adipate, 2-hexyldecyl adipate, di-2-heptylundecyl adipate, monoisostearic acid N-alkyl glycol, isocetyl isostearate, trimethylolpropane triisostearate, ethylene glycol di-2-ethyl hexanoate, cetyl 2-ethylhexanoate, trimethylolpropane tri-2-ethylhexanoate, pentaerythritol tetra-2-ethylhexanoate, cetyl octanoate, octyldodecyl gum ester, oleyl oleate, octyldodecyl oleate, decyl oleate, neopentyl glycol dicaprate, triethyl citrate, 2-ethylhexyl succinate, isocetyl stearate, butyl stearate, diisopropyl sebacate, di-2-ethylhexyl sebacate, cetyl lactate, myristyl lactate, isopropyl palmitate, 2-ethylhexyl palmitate, 2-hexyldecyl palmitate, 2-heptylundecyl palmitate, cholesteryl 12-hydroxystearate, dipentaerythritol fatty acid ester, isopropyl myristate, octyldodecyl myristate, 2-hexyldecyl myristate, myristyl myristate, hexyldecyl dimethyloctanoate, ethyl laurate, hexyl laurate, diisostearyl malate, dicaprylyl carbonate, and mixtures thereof. Nonetheless, in some cases, the nanoemulsion preferably includes at least isopropyl myristate as the one or more oils.

Moreover, in some instances, the ester oil is preferably selected from octyldodecyl myristate, isostearyl palmitate, hexyldecyl isostearate, oleyl oleate, isocetyl myristate, stearyl stearate, decyl oleate, ethylhexyl stearate, cetyl caprate, octyl palmitate, cetyl 2-ethylhexanoate, isopropyl isostearate, hexyl laurate, isopropyl palmitate, isopropyl linoleate, and isopropyl myristate, and a mixture thereof.

The one or more oils are in the form of nanoemulsion droplets having an average size of about 20 nm to about 500 nm. The size of the nanoemulsion droplets, however, can vary. Therefore, in some instances, the size of the nanoemulsion droplets are about 40 nm to about 500 nm, about 50 nm to about 500 nm, about 20 nm to about 400 nm, about 40 nm to about 400 nm, about 50 nm to about 400 nm, about 20 nm to about 300 nm, about 40 nm to about 300 nm, about 50 nm to about 300 nm, about 20 nm to about 200 nm, about 40 nm to about 200 nm, or about 50 nm to about 200 nm.

The one or more surfactants may have a hydrophilic-lipophilic balance (HLB) of about 11 to about 16. When a combination of two or more surfactants are included the nanoemulsion, the combination of surfactants typically has an HLB of about 11 to about 16. For example, a combination of two surfactants may be included, wherein one of the surfactants has an HLB below 11 and one of the surfactants has an HLB of above 16. Nonetheless, in combination, the HLB is from about 11 to about 16. In some instances, the one or more surfactants (or combination of surfactants) has an HLB of about 11 to about 15, about 11 to about 14, about 11 to about 13, about 12 to about 16, about 12 to about 15, or about 12 to about 14.

The total amount of oil(s) may vary but is typically about 10 to about 50 wt. %, based on the total weight of the nanoemulsion. In some instances, the total amount of the oil(s) may be from about 10 to about 45 wt. %, about 10 to about 40 wt. %, about 10 to about 30 wt. %, about 10 to about 25 wt. %, about 15 to about 50 wt. %, about 15 to about 45 wt. %, about 15 to about 40 wt. %, about 15 to about 35 wt. %, about 15 to about 30 wt. %, or about 15 to about 25 wt. %, based on the total weight of the nanoemulsion.

Surfactants

The various compositions described herein may include one or more surfactants, including cationic, anionic, non-ionic and/or amphoteric/zwitterionic surfactants. The total amount of surfactants in the nanoemulsion may vary but is typically about 1 to about 25 wt. %, based on the total weight of the nanoemulsion. In some cases, the total amount of surfactants is about 1 to about 20 wt. %, about 1 to about 15 wt. %, about 1 to about 10 wt. %, about 2 to about 25 wt. %, about 2 to about 20 wt. %, about 2 to about 15 wt. %, about 2 to about 10 wt. %, about 5 to about 25 wt. %, about 5 to about 20 wt. %, about 5 to about 15 wt. %, or about 5 to about 10 wt. %, based on the total weight of the nanoemulsion.

Non-limiting examples of surfactants that may be used are provided below.

a. Nonionic Surfactants

Examples of nonionic surfactants that may be used are described, for example, in the Handbook of Surfactants by M. R. Porter, published by Blackie & Son (Glasgow and London), 1991, pp. 116-178, which is incorporated herein by reference in its entirety. Non-limiting examples of nonionic surfactants include those selected from polyol esters, glycerol ethers, oxyethylenated ethers, oxypropylenated ethers, and ethylene glycol polymers. More specific but non-limiting examples include sorbitan fatty esters (e.g., sorbitan oleate), ethoxylated sorbitan fatty esters (e.g., a polysorbate), and a mixture thereof.

In some instances, PEG-sorbitan fatty acid esters are particularly well suited for use as nonionic surfactants. Non-limiting examples include PEG-20 sorbitan monolaurate (Tween-20), PEG-20 sorbitan monopalmitate (Tween-40), PEG-20 sorbitan monostearate (Tween-60), and PEG-20 sorbitan monooleate (Tween-80).

I some instances, sorbitan esters of fatty acids are particularly well suited for use as nonionic surfactants. Non-limiting examples include sorbitan monolaurate (Arlacel 20), sorbitan monopalmitate (Span-40), sorbitan monooleate (Span-80), sorbitan monostearate, and sorbitan tristearate.

Furthermore, in some cases, it is preferable to include one or more PEG_sorbitan fatty acid esters and one or more sorbitan esters of fatty acids, for example, a polysorbate (e.g., polysorbate-80) and sorbitan monoleate).

Although polyethylene glycol (PEG) itself may not function as a surfactant (it functions as a co-surfactant), a variety of PEG-fatty acid esters are useful nonionic surfactants. Exemplary monoesters include esters of lauric acid, oleic acid, and stearic acid, e.g., PEG-8 laurate, PEG-8 oleate, PEG-8 stearate, PEG-9 oleate, PEG-10 laurate, PEG-10 oleate, PEG-12 laurate, PEG-12 oleate, PEG-15 oleate, PEG-20 laurate and PEG-20 oleate. Polyethylene glycol fatty acid diesters suitable for use as non-ionic surfactants in the compositions of the present invention include PEG-20 dilaurate, PEG-20 dioleate, PEG-20 distearate, PEG-32 dilaurate and PEG-32 dioleate. Suitable polyethylene glycol glycerol fatty acid esters include PEG-20 glyceryl laurate, PEG-30 glyceryl laurate, PEG-40 glyceryl laurate, PEG-20 glyceryl oleate, and PEG-30 glyceryl oleate.

A large number of surfactants of different degrees of hydrophobicity or hydrophilicity can be prepared by reaction of alcohols or polyalcohols with a variety of natural and/or hydrogenated oils. Most commonly, the oils used are castor oil or hydrogenated castor oil, or an edible vegetable oil such as corn oil, olive oil, peanut oil, palm kernel oil, apricot kernel oil, or almond oil. Preferred alcohols include glycerol, propylene glycol, ethylene glycol, polyethylene glycol, sorbitol, and pentaerythritol. Among these alcohol-oil transesterified surfactants, preferred hydrophilic surfactants are PEG-35 castor oil (Incrocas-35), PEG-40 hydrogenated castor oil (Cremophor RH 40), PEG-25 trioleate (TAGAT TO), PEG-60 corn glycerides (Crovol M70), PEG-60 almond oil (Crovol A70), PEG-40 palm kernel oil (Crovol PK70), PEG-50 castor oil (Emalex C-50), PEG-50 hydrogenated castor oil (Emalex HC-50), PEG-8 caprylic/capric glycerides (Labrasol), and PEG-6 caprylic/capric glycerides (Softigen 767). Preferred hydrophobic surfactants in this class include PEG-5 hydrogenated castor oil, PEG-7 hydrogenated castor oil, PEG-9 hydrogenated castor oil, PEG-6 corn oil (LABRAFIL M 2125 CS), PEG-6 almond oil (LABRAFIL M 1966 CS), PEG-6 apricot kernel oil (LABRAFIL M 1944 CS), PEG-6 olive oil (LABRAFIL M 1980 CS), PEG-6 peanut oil (LABRAFIL M 1969 CS), PEG-6 hydrogenated palm kernel oil (LABRAFIL M 2130 BS), PEG-6 palm kernel oil (LABRAFIL M 2130 CS), PEG-6 triolein (LABRAFI b M 2735 CS), PEG-8 corn oil (LABRAFIL WL 2609 BS), PEG-20 corn glycerides (CROVOL M40), and PEG-20 almond glycerides (CROVOL A40).

Polyglycerol esters of fatty acids are also suitable nonionic surfactants. Among the polyglyceryl fatty acid esters, examples include polyglyceryl oleate (Plurol Oleique), polyglyceryl-2 dioleate (NIKKOL DGDO), and polyglyceryl-10 trioleate.

Ethers of polyethylene glycol and alkyl alcohols are suitable non-ionic surfactants for use in the present invention. Exemplary hydrophobic ethers include PEG-3 oleyl ether (Volpo 3) and PEG-4 lauryl ether (Brij 30).

Esters of lower alcohols (C₂ to C₄) and fatty acids (C₈ to C₁₈) are suitable non-ionic surfactants for use in the present invention. Among these esters, preferred hydrophobic surfactants include ethyl oleate (CRODAMOL EO), isopropyl myristate (CRODAMOL IPM), and isopropyl palmitate (CRODAMOL IPP).

The nonionic surfactant may be alcohols, alpha-diols and (C₁-C₂₄)alkylphenols, these compounds being polyethoxylated, polypropoxylated and/or polyglycerolated, and containing at least one fatty chain comprising, for example, from 8 to 18 carbon atoms, it being possible for the number of ethylene oxide and/or propylene oxide groups to especially range from 2 to 50, and for the number of glycerol groups to especially range from 2 to 30.

Mention may also be made of copolymers of ethylene oxide and propylene oxide, optionally oxyethylenated sorbitan fatty acid esters, sucrose fatty acid esters, polyoxyalkylenated fatty acid esters, polyoxyalkylenated fatty amides, optionally oxyalkylenated alkyl(poly)glucosides, alkylglucoside esters, derivatives of N-alkylglucamine and of N-acylmethylglucamine, aldobionamides, amine oxides and (poly)oxyalkylenated silicones.

The nonionic surfactants are more particularly chosen from monooxyalkylenated or polyoxyalkylenated and monoglycerolated or polyglycerolated nonionic surfactants, and alkyl(poly)glucosides. The oxyalkylene units are more particularly oxyethylene or oxypropylene units, or a combination thereof, preferably oxyethylene units.

Useful nonionic surfactants may include: oxyalkylenated (C₈-C₂₄)alkylphenols; saturated or unsaturated, linear or branched, oxyalkylenated C₈-C₄₀ alcohols; saturated or unsaturated, linear or branched, oxyalkylenated C₈-C₃₀ amides; esters of saturated or unsaturated, linear or branched, C₈-C₃₀ acids and of polyethylene glycols; saturated or unsaturated, oxyethylenated plant oils; condensates of ethylene oxide and/or of propylene oxide, alone or as mixtures; oxyethylenated and/or oxypropylenated silicones; and alkyl(poly)glucosides.

As examples of monoglycerolated or polyglycerolated nonionic surfactants, monoglycerolated or polyglycerolated C₈-C₄₀ alcohols are useable. In particular, the monoglycerolated or polyglycerolated C C₈-C₄₀ alcohols correspond to formula (VIII) below:

R₂₉O—[CH₂—CH(CH₂OH)—O]ₘ—H (VIII)

in which formula (VIII):

R₂₉ represents a linear or branched C₈-C₄₀ and preferably C₈-C₃₀ alkyl or alkenyl radical; and

m represents a number ranging from 1 to 30, or from 1 to 10.

As examples of compounds of formula (VIII), mention may be made of lauryl alcohol containing 4 mol of glycerol (INCI name: Polyglyceryl-4 Lauryl Ether), lauryl alcohol containing 1.5 mol of glycerol, oleyl alcohol containing 4 mol of glycerol (INCI name: Polyglyceryl-4 Oleyl Ether), oleyl alcohol containing 2 mol of glycerol (INCI name: Polyglyceryl-2 Oleyl Ether), cetearyl alcohol containing 2 mol of glycerol, cetearyl alcohol containing 6 mol of glycerol, oleocetyl alcohol containing 6 mol of glycerol, and octadecanol containing 6 mol of glycerol.

The alcohol of formula (VIII) may represent a mixture of alcohols in the same way that the value of m represents a statistical value, which means that, in a commercial product, several species of polyglycerolated fatty alcohols may coexist in the form of a mixture.

The alkyl(poly)glycoside nonionic surfactant(s) may be represented by formula (IX) below:

R₃₀O—(R₃₁O)ₜ(G)ᵥ (IX)

in which:

R₃₀ represents a saturated or unsaturated, linear or branched alkyl group comprising from about 8 to 24 carbon atoms, or an alkylphenyl group in which the linear or branched alkyl group comprises from 8 to 24 carbon atoms;

R₃₁ represents an alkylene group containing from about 2 to 4 carbon atoms,

G represents a saccharide unit comprising from 5 to 6 carbon atoms,

t denotes a value ranging from 0 to 10, or from 0 to 4, and

v denotes a value ranging from 1 to 15.

In some cases, the alkyl(poly)glycoside nonionic surfactant(s) correspond to formula (IX) in which:

R₃₀ denotes a linear or branched, saturated or unsaturated alkyl group containing from 8 to 18 carbon atoms,

G denotes glucose, fructose or galactose, preferably glucose,

t denotes a value ranging from 0 to 3, and is preferably equal to 0, and

R₃₁ and v are as defined previously.

The degree of polymerization of the alkyl(poly)glucoside nonionic surfactant(s), as represented, for example, by the index v in formula (IX), ranges on average from 1 to 15, or from 1 to 4. This degree of polymerization more particularly ranges from 1 to 2 and better still from 1.1 to 1.5, on average.

The glycoside bonds between the saccharide units are of 1.6 or 1.4 type and preferably of 1.4 type.

Examples of compounds of formula (IX) that may especially be mentioned are the products sold by the company Cognis under the names Plantaren® (600 CS/U, 1200 and 2000) or Plantacare® (818, 1200 and 2000). Use may also be made of the products sold by the company SEPPIC under the names Triton CG 110 (or Oramix CG 110) and Triton CG 312 (or Oramix® NS 10), the products sold by the company BASF under the name Lutensol GD 70 or the products sold by the company Chem Y under the name AG10 LK. Use may also be made, for example, of the 1,4-(C₈-C₁₆) alkylpolyglucoside as an aqueous solution at 53% by weight relative to the total weight of the solution, sold by Cognis under the reference Plantacare 818 UP.

The total amount of nonionic surfactants in the nanoemulsion, if present, may vary but are is typically about 1 to about 25 wt. %, based on the total weight of the nanoemulsion. In some cases, the total amount of nonionic surfactants is about 1 to about 20 wt. %, about 1 to about 15 wt. %, about 1 to about 10 wt. %, about 2 to about 25 wt. %, about 2 to about 20 wt. %, about 2 to about 15 wt. %, about 2 to about 10 wt. %, about 5 to about 25 wt. %, about 5 to about 20 wt. %, about 5 to about 15 wt. %, or about 5 to about 10 wt. %, based on the total weight of the nanoemulsion.

b. Amphoteric or Zwitterionic Surfactants

The amphoteric or zwitterionic surfactant that may be used in compositions according to the disclosure may be derivatives of aliphatic secondary or tertiary amines, optionally quaternized, in which derivatives the aliphatic group is a linear or branched chain comprising from 8 to 22 carbon atoms, the amine derivatives containing at least one anionic group, such as a carboxylate, sulfonate, sulfate, phosphate or phosphonate group. Mention may be made in particular of (C₈-C₂₀)alkylbetaines such as cocoylbetaine, sulfobetaines, (C₈-C₂₀)alkylamido(C₂-C₈)alkylbetaines such as cocoylamidopropylbetaine or (C₈-C₂₀)alkylamido(C₆-C₈)-alkylsulfobetaines, and mixtures thereof.

Among the derivatives of aliphatic secondary or tertiary amines, optionally quaternized, that may be used, as defined above, mention may also be made of the compounds of respective structures (I), (II) and (IIa) below:

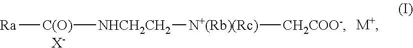

in which formula (I):

Ra represents a C₁₀-C₃₀ alkyl or alkenyl group derived from an acid Ra—COOH preferably present in hydrolysed coconut oil, or a heptyl, nonyl or undecyl group; Rb represents a beta-hydroxyethyl group; and

Rc represents a carboxymethyl group;

M⁺ represents a cationic counterion derived from an alkali metal or alkaline-earth metal, such as sodium, an ammonium ion or an ion derived from an organic amine; and

X⁻ represents an organic or mineral anionic counterion, such as that chosen from halides, acetates, phosphates, nitrates, (C₁-C₄)alkyl sulfates, (C₁-C₄)alkyl- or (C₁-C₄)alkylarylsulfonates, in particular methyl sulfate and ethyl sulfate; or alternatively M⁺ and X⁻ are absent;

Ra′—C(O)—NHCH₂CH₂—N(B)(B′) (II)

in which formula (II):

B represents the group —CH₂—CH₂—O—X′;

B′ represents the group —(CH²)_{z}Y′, with z=1 or 2;

X′ represents the group —CH₂—COOH, CH₂—COOZ′, —CH₂CH₂—COOH or —CH₂CH₂—COOZ′, or a hydrogen atom;

Y′ represents the group —COOH, —COOZ′, CH₂CH(OH)SO₃H or the group —CH₂CH(OH)SO₃Z′;

Z′ represents a cationic counterion derived from an alkali metal or alkaline-earth metal, such as sodium, an ammonium ion or an ion derived from an organic amine;

Ra′ represents a C₁₀-C₃₀ alkyl or alkenyl group of an acid Ra′—COOH, which may be coconut oil or in hydrolysed linseed oil, or an alkyl group, especially a C₁₇ group and its iso form, or an unsaturated C₁₇ group.

The compounds of this type are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by the company Rhodia under the trade name Miranol C2M Concentrate and the cocoamphodipropionate sold by the company Evonik Goldschmidt under the trade name Rewoteric AM KSF 40.

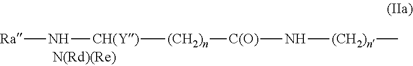

in which formula (IIa):

Y″ represents the group —COOH, —COOZ″, —CH₂CH(OH)SO₃H or the group —CH₂CH(OH)SO₃Z″;

Rd and Re, independently of each other, represent a C₁-C₄ alkyl or hydroxyalkyl radical;

Z″ represents a cationic counterion derived from an alkali metal or alkaline-earth metal, such as sodium, an ammonium ion or an ion derived from an organic amine;

Ra″ represents a C₁₀-C₃₀ alkyl or alkenyl group of an acid Ra″—COOH;

n and n′ denote, independently of each other, an integer ranging from 1 to 3;

and mixtures of these compounds.

Among the compounds of formula (IIa), mention may be made of the compound classified in the CTFA dictionary under the name sodium diethylaminopropyl cocoaspartamide and sold by the company Chimex under the name Chimexane HB. In some instances, the amphoteric or zwitterionic surfactant(s) are chosen from cocoylbetaine, cocoylamidopropylbetaine and sodium cocoylamidoethyl-N-hydro xyethylaminopropionate.

The total amount of amphoteric surfactants in the nanoemulsion, if present, may vary but is typically about 0.1 to about 20 wt. %, based on the total weight of the nanoemulsion. In some instances, the total amount of amphoteric surfactants may be about 0.1 to about 15 wt. %, about 0.1 to about 10 wt. %, about 0.1 to about 8 wt. %, about 0.1 to about 5 wt. %, about 1 to about 20 wt. %, about 1 to about 15 wt. %, about 1 to about 10 wt. %, about 1 to about 8 wt. %, or about 1 to about 5 wt. %, based on the total weight of the nanoemulsion.

c. Cationic Surfactants

The term “cationic surfactant” means a surfactant that is positively charged when it is contained in the composition according to the disclosure. This surfactant may bear one or more positive permanent charges or may contain one or more functions that are cationizable in the composition according to the disclosure.

Non-limiting examples of cationic surfactants include behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride (Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

The cationic surfactant(s) may be chosen from optionally polyoxyalkylenated, primary, secondary or tertiary fatty amines, or salts thereof, and quaternary ammonium salts, and mixtures thereof.

The fatty amines generally comprise at least one C₈-C₃₀ hydrocarbon-based chain.

Examples of quaternary ammonium salts that may especially be mentioned include: those corresponding to the general formula (III) below:

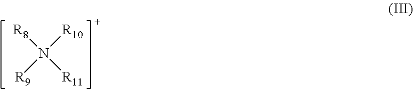

in which the groups R₈ to R₁₁, which may be identical or different, represent a linear or branched, saturated or unsaturated aliphatic group comprising from 1 to 30 carbon atoms, or an aromatic group such as aryl or alkylaryl, at least one of the groups R₈ to R₁₁ denoting a group comprising from 8 to 30 carbon atoms and preferably from 12 to 24 carbon atoms. The aliphatic groups may comprise heteroatoms especially such as oxygen, nitrogen, sulfur and halogens. The aliphatic groups are chosen, for example, from C₁-C₃₀ alkyl, C₂-C₃₀ alkenyl, C₁-C₃₀ alkoxy, polyoxy(C₂-C₆)alkylene, C₁-C₃₀ alkylamide, (C₁₂-C₂₂)alkylamido(C₂-C₆)alkyl, (C₁₂-C₂₂)alkyl acetate and C₁-C₃₀ hydroxyalkyl groups; X is an anion chosen from the group of halides, phosphates, acetates, lactates, (C₁-C₄)alkyl sulfates, and (C₁-C₄)alkyl- or (C₁-C₄)alkylarylsulfonates.

Among the quaternary ammonium salts of formula (III), those that are preferred are, on the one hand, tetraalkylammonium salts, for instance dialkyldimethylammonium or alkyltrimethylammonium salts in which the alkyl group contains approximately from 12 to 22 carbon atoms, in particular behenyltrimethylammonium, distearyldimethylammonium, cetyltrimethylammonium or benzyldimethylstearylammonium salts, or, on the other hand, oleocetyldimethylhydroxyethylammonium salts, palmitylamidopropyltrimethylammonium salts, stearamidopropyltrimethylammonium salts and stearamidopropyldimethylcetearylammonium salts.

In some cases it is useful to use salts such as the chloride salts of the following compounds:

A. a quaternary ammonium salt of imidazoline, such as, for example, those of formula (IV) below:

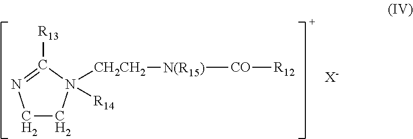

in which R₁₂ represents an alkenyl or alkyl group comprising from 8 to 30 carbon atoms, derived for example from tallow fatty acids, R₁₃ represents a hydrogen atom, a C₁-C₄ alkyl group or an alkyl or alkenyl group comprising from 8 to 30 carbon atoms, R₁₄ represents a C₁-C₄ alkyl group, R₁₅ represents a hydrogen atom or a C₁-C₄ alkyl group, X⁻ is an anion chosen from the group of halides, phosphates, acetates, lactates, alkyl sulfates, alkyl- or alkylaryl-sulfonates in which the alkyl and aryl groups preferably comprise, respectively, from 1 to 20 carbon atoms and from 6 to 30 carbon atoms. R₁₂ and R₁₃ preferably denote a mixture of alkenyl or alkyl groups containing from 12 to 21 carbon atoms, derived for example from tallow fatty acids, R₁₄ preferably denotes a methyl group, and R₁₅ preferably denotes a hydrogen atom. Such a product is sold, for example, under the name Rewoquat® W 75 by the company Rewo;

B. a quaternary diammonium or triammonium salt, in particular of formula (V):

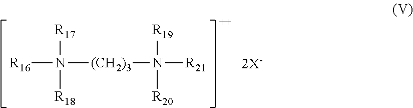

in which R₁₆ denotes an alkyl radical comprising approximately from 16 to 30 carbon atoms, which is optionally hydroxylated and/or interrupted with one or more oxygen atoms, R₁₇ is chosen from hydrogen or an alkyl radical comprising from 1 to 4 carbon atoms or a group (R₁₆ₐ)(R₁₇ₐ)(R₁₈ₐ)N—(CH₂)₃,

R₁₆ₐ, R₁₇ₐ, R₁₈ₐ, R₁₈, R₁₉, R₂₀ and R₂₁, which may be identical or different, being chosen from hydrogen and an alkyl radical comprising from 1 to 4 carbon atoms, and X⁻ is an anion chosen from the group of halides, acetates, phosphates, nitrates and methyl sulfates. Such compounds are, for example, Finquat CT-P, sold by the company Finetex (Quaternium 89), and Finquat CT, sold by the company Finetex (Quaternium 75),

C. a quaternary ammonium salt containing at least one ester function, such as those of formula (VI) below:

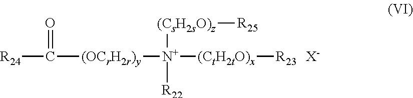

in which:

R₂₂ is chosen from C₁-C₆ alkyl groups and C₁-C₆ hydroxyalkyl or dihydroxyalkyl groups;

R₂₃ is chosen from:

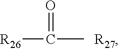

which is a linear or branched, saturated or unsaturated C₁-C₂₂ hydrocarbon-based group, and a hydrogen atom,

R₂₅ is chosen from:

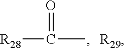

which is a linear or branched, saturated or unsaturated C₁-C₆ hydrocarbon-based group, and a hydrogen atom,

R₂₄, R₂₆ and R₂₈, which may be identical or different, are chosen from linear or branched, saturated or unsaturated C₇-C₂₁ hydrocarbon-based groups;

r, s and t, which may be identical or different, are integers ranging from 2 to 6;

y is an integer ranging from 1 to 10;

x and z, which may be identical or different, are integers ranging from 0 to 10;

X⁻ is a simple or complex, organic or mineral anion;

with the proviso that the sum x+y+z is from 1 to 15, that when x is 0 then Rₙ denotes R₂₇, and that when z is 0 then R₂₅ denotes R₂₉.

The alkyl groups R₂₂ may be linear or branched, and more particularly linear. In some cases, R₂₂ denotes a methyl, ethyl, hydroxyethyl or dihydroxypropyl group, and more particularly a methyl or ethyl group. Advantageously, the sum x+y+z is from 1 to 10.

When R₂₃ is a hydrocarbon-based group R₂₇, it may be long and contain from 12 to 22 carbon atoms, or may be short and contain from 1 to 3 carbon atoms. When R₂₅ is an R₂₉ hydrocarbon-based group, it preferably contains 1 to 3 carbon atoms. Advantageously, R₂₄, R₂₆ and R₂₈, which may be identical or different, are chosen from linear or branched, saturated or unsaturated C₁₁-C₂₁ hydrocarbon-based groups, and more particularly from linear or branched, saturated or unsaturated C₁₁-C₂₁ alkyl and alkenyl groups.

In some cases, x and z, which may be identical or different, have values of 0 or 1. Likewise, in some cases y is equal to 1. In some cases, r, s and t, which may be identical or different, are equal to 2 or 3, and even more particularly are equal to 2.

The anion X⁻ is may be a halide (chloride, bromide or iodide) or an alkyl sulfate, more particularly methyl sulfate. However, use may be made of methanesulfonate, phosphate, nitrate, tosylate, an anion derived from an organic acid, such as acetate or lactate, or any other anion compatible with the ammonium containing an ester function.

The anion X⁻ is even more particularly chloride or methyl sulfate.

Use is made more particularly, in the composition according to the invention, of the ammonium salts of formula (VI) in which:

R₂₂ denotes a methyl or ethyl group,

x and y are equal to 1;

z is equal to 0 or 1;

r, s and t are equal to 2;

R₂₃ is chosen from:

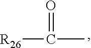

methyl, ethyl or C₁₄-C₂₂ hydrocarbon-based groups, and a hydrogen atom;

R₂₅ is chosen from:

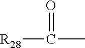

and a hydrogen atom;

R₂₄, R₂₆ and R₂₈, which may be identical or different, are chosen from linear or branched, saturated or unsaturated C₁₃-C₁₇ hydrocarbon-based groups, and preferably from linear or branched, saturated or unsaturated C₁₃-C₁₇ alkyl and alkenyl groups. The hydrocarbon-based groups are advantageously linear.

Mention may be made, for example, of the compounds of formula (VI) such as the diacyloxyethyldimethylammonium, diacyloxyethylhydroxyethylmethylammonium, monoacyloxyethyldihydroxyethylmethylammonium, triacyloxyethylmethylammonium and monoacyloxyethylhydroxyethyldimethylammonium salts (chloride or methyl sulfate in particular), and mixtures thereof. The acyl groups preferably contain 14 to 18 carbon atoms and are obtained more particularly from a plant oil, such as palm oil or sunflower oil. When the compound contains several acyl groups, these groups may be identical or different.

These products are obtained, for example, by direct esterification of triethanolamine, triisopropanolamine, an alkyldiethanolamine or an alkyldiisopropanolamine, which are optionally oxyalkylenated, with C₁₀-C₃₀ fatty acids or with mixtures of C₁₀-C₃₀ fatty acids of plant or animal origin, or by transesterification of the methyl esters thereof. This esterification is followed by quaternization using an alkylating agent such as an alkyl (preferably methyl or ethyl) halide, a dialkyl (preferably methyl or ethyl) sulfate, methyl methanesulfonate, methyl para-toluenesulfonate, glycol chlorohydrin or glycerol chlorohydrin. Such compounds are, for example, sold under the names Dehyquart® by the company Henkel, Stepanquat® by the company Stepan, Noxamium® by the company Ceca or Rewoquat® WE 18 by the company Rewo-Witco.

The composition according to the invention may contain, for example, a mixture of quaternary ammonium monoester, diester and triester salts with a weight majority of diester salts.

The total amount of cationic surfactants in the nanoemulsion, if present, may vary but is typically about 0.1 to about 20 wt. %, based on the total weight of the nanoemulsion. In some instances, the total amount of cationic surfactants may be about 0.1 to about 15 wt. %, about 0.1 to about 10 wt. %, about 0.1 to about 8 wt. %, about 0.1 to about 5 wt. %, about 1 to about 20 wt. %, about 1 to about 15 wt. %, about 1 to about 10 wt. %, about 1 to about 8 wt. %, or about 1 to about 5 wt. %, based on the total weight of the nanoemulsion.

d. Anionic Surfactants

The term “anionic surfactant” means a surfactant comprising, as ionic or ionizable groups, only anionic groups. These anionic groups are chosen preferably from the groups CO₂H, CO₂⁻, SO₃H, SO₃⁻, OSO₃H, OSO₃⁻O₂PO₂H, O₂PO₂H and O₂PO₂²⁻.

The anionic surfactant(s) that may be used may be alkyl sulfates, alkyl ether sulfates, alkylamido ether sulfates, alkylaryl polyether sulfates, monoglyceride sulfates, alkylsulfonates, alkylamide sulfonates, alkylarylsulfonates, alpha-olefin sulfonates, paraffin sulfonates, alkylsulfosuccinates, alkyl ether sulfosuccinates, alkylamide sulfosuccinates, alkyl sulfoacetates, acylsarcosinates, acylglutamates, alkylsulfosuccinamates, acylisethionates and N-acyltaurates, salts of alkyl monoesters and polyglycoside-polycarboxylic acids, acyllactylates, salts of D-galactoside uronic acids, salts of alkyl ether carboxylic acids, salts of alkyl aryl ether carboxylic acids, and salts of alkylamido ether carboxylic acids; or the non-salified forms of all of these compounds, the alkyl and acyl groups of all of these compounds containing from 6 to 24 carbon atoms and the aryl group denoting a phenyl group. Some of these compounds may be oxyethylenated and then preferably comprise from 1 to 50 ethylene oxide units.

The salts of C₆-C₂₄ alkyl monoesters of polyglycoside-polycarboxylic acids may be chosen from C₆-C₂₄ alkyl polyglycoside-citrates, C₆-C₂₄ alkyl polyglycoside-tartrates and C₆-C₂₄ alkyl polyglycoside-sulfo succinates.

When the anionic surfactant(s) are in salt form, they may be chosen especially from alkali metal salts such as the sodium or potassium salt and preferably the sodium salt, ammonium salts, amine salts and in particular amino alcohol salts, or alkaline-earth metal salts such as the magnesium salt.

Examples of amino alcohol salts that may especially be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts. Alkali metal or alkaline-earth metal salts and in particular the sodium or magnesium salts may be used.

Use is also made of (C₆-C₂₄)alkyl sulfates, (C₆-C₂₄)alkyl ether sulfates, which are optionally ethoxylated, comprising from 2 to 50 ethylene oxide units, and mixtures thereof, in particular in the form of alkali metal salts or alkaline-earth metal salts, ammonium salts or amino alcohol salts. More preferentially, the anionic surfactant(s) are chosen from (C₁₀-C₂₀)alkyl ether sulfates, and in particular sodium lauryl ether sulfate containing 2.2 mol of ethylene oxide.

The total amount of anionic surfactants in the nanoemulsion, if present, may vary but is typically about 0.1 to about 20 wt. %, based on the total weight of the nanoemulsion. In some instances, the total amount of anionic surfactants may be about 0.1 to about 15 wt. %, about 0.1 to about 10 wt. %, about 0.1 to about 8 wt. %, about 0.1 to about 5 wt. %, about 1 to about 20 wt. %, about 1 to about 15 wt. %, about 1 to about 10 wt. %, about 1 to about 8 wt. %, or about 1 to about 5 wt. %, based on the total weight of the nanoemulsion.

Nonionic Co-Surfactants

As used herein, the term “co-surfactant” refers to a compound that enhances the efficacy of a surfactant. In some embodiments, the co-surfactant is not amphiphilic (i.e., may be all hydrophobic or hydrophilic). In further embodiments, the co-surfactant is hydrophilic. Although not wishing to be bound by any particular theory, it is thought that the co-surfactant interacts with the surfactant such that it adjusts the packing of the surfactant at the oil/water interface and allows the surfactant to act more efficiently.

Numerous other nonionic co-surfactants are disclosed in McCutcheon's Detergents and Emulsifiers, 1993 Annuals, published by McCutcheon Division, MC Publishing Co., Glen Rock, N.J., pp. 1-246 and 266-273; in the CTFA International Cosmetic Ingredient Dictionary, Fourth Ed., Cosmetic, Toiletry and Fragrance Association, Washington, D.C. (1991) (hereinafter the CTFA Dictionary) at pages 1-651; and in the CTFA Cosmetic Ingredient Handbook, First Ed., Cosmetic, Toiletry and Fragrance Association, Washington, D.C. (1988) (hereafter the CTFA Handbook), at pages 86-94, each incorporated herein by reference.

In some instances, the one or more nonionic co-surfactants are selected from polyalkoxylated co-surfactants. For example, the nonionic co-surfactant may comprise at least one chain formed of ethylene oxide units or ethylene oxide and propylene oxide units. The co-surfactant may, for example, be selected from the conjugated compounds polyethylene glycol/phosphatidylethanolamine (PEG/PE), fatty acid and polyethylene glycol ethers and fatty acid and polyethylene glycol esters, and ethylene oxide and propylene oxide block copolymers.

In particular, the nonionic co-surfactant may be selected from polyethylene glycols having a molecular weight of 150 to 1000, polypropylene glycol of the formula HO(CH₃CHCH₂O)ₙH, wherein n is 2 to 18, mixtures of polyethylene glycol and polypropylene glycol, mono and di C₁-C₆ alkyl ethers and esters of ethylene glycol and propylene glycol having the formulas of R(X)ₙOH and R₁(X)ₙOH, R(X)ₙOR, R₁(X)ₙOR₁ and R₁(X)ₙOR wherein R is a C₁₋₆ alkyl group, R₁ is a C₂₋₄ acyl group, X is (OCH₂ CH₂) or (OCH₂ CHCH₃) and n is from 1 to 4.

In some instances, the nonionic co-surfactant is preferably a polyethylene glycol depicted by the formula:

HO(CH₂—CH₂—)ₙH

wherein n is about 8 to about 225, preferably about 10 to about 150, more preferably about 10 to about 100, wherein PEG600 or PEG400 are preferred (which are polyethylene glycols having a molecular weight of about 400 and about 600).

The total amount of co-surfactant(s) in the nanoemulsion may vary but is typically about 0.1 to about 10 wt. %, based on the total weight of the nanoemulsion. In some instances, the total amount of co-surfactant(s) in the nanoemulsion is about 0.1 to about 8 wt. %, about 0.1 to about 6 wt. %, about 0.5 to about 10 wt. %, about 0.5 to about 8 wt. %, about 0.5 to about 6 wt. %, about 1 to about 10 wt. %, about 1 to about 8 wt. %, or about 1 to about 6 wt. %, based on the total weight of the nanoemulsion.

Water

The total amount of water in the nanoemulsion may vary but is typically about 25 to about 75 wt. %, based on the total weight of the nanoemulsion. In some cases, the total amount of water is about 30 to about 75 wt. %, about 35 to about 75 wt. %, about 40 to about 75 wt. %, about 30 to about 70 wt. %, about 30 to about 60 wt. %, about 35 to about 70 wt. %, about 40 to about 70 wt. %, or about 40 to about 60 wt. %, based on the total weight of the nanoemulsion.

Lipophilic Active Agents

The term “lipophilic active ingredient” (or “lipophilic active agent”) refers to an active agent that is capable of being dissolved in a fatty phase, in particular the oil droplets of the nanoemulsion.

Non-limiting examples of lipophilic active ingredients which can be used in accordance with the disclosure include organic UV screening agents such as para-aminobenzoic acid derivatives, salicylic derivatives, cinnamic derivatives, benzophenones and aminobenzophenones, anthranillic derivatives, β, β-diphenylacrylate derivatives, benzylidenecamphor derivatives, phenylbenzimidazole derivatives, benzotriazole derivatives, triazine derivatives, bisresorcinyl triazines, imidazoline derivatives, benzalmalonate derivatives, 4,4-diarylbutadiene derivatives, benzoxazole derivatives, merocyanines, malonitrile or malonate diphenyl butadiene derivatives, chalcones and mixtures thereof.

In some instance, the nanoemulsions include one or more UV filtering agents (also referred to as “UV filters”), in particular, one or more organic UV filtering agents. UV filtering agents are well known in the art for their use in stopping UV radiation. Non-limiting examples of UV filters include:
i. Sparingly soluble UV filters (not appreciably soluble in either water or oil) such as Methylene Bis-Benzotriazolyl Tetramethylbutylphenol, Tris-Biphenyl Triazine, Methanone, 1,1′-(1,4-piperazinediyl)bis[1-[2-[4-(diethylamino)-2-hydroxybenzoyl]phen-yl]- and mixtures thereof.
ii. Oil soluble organic UV filters (at least partially soluble in oil or organic solvent), such as Bis-Ethylhexyloxyphenol Methoxyphenyl Triazine, Butyl Methoxydibenzoylmethane (BMBM), Oxybenzone, Sulisobenzone, Diethylhexyl Butamido Triazone (DBT), Drometrizole Trisiloxane, Ethylhexyl Methoxycinnamate (EHMC), Ethylhexyl Salicylate (EHS), Ethylhexyl Triazone (EHT), Homosalate, Isoamyl p-Methoxycinnamate, 4-Methylbenzylidene Camphor, Octocrylene (OCR), Polysilicone-15, and Diethylamino Hydroxy Benzoyl Hexyl Benzoate (DHHB);
iii. Inorganic UV filters such as titanium oxide and zinc oxide, iron oxide, zirconium oxide and cerium oxide; and
iv. Water soluble UV filters such as Phenylbenzimidazole Sulfonic Acid (PBSA), Sulisobenzone-sodium salt, Benzydilene Camphor Sulfonic Acid, Camphor Benzalkonium Methosulfate, Cinoxate, Disodium Phenyl Dibenzylmidazole Tetrasulfonate, Terephthalylidene Dicamphor Sulfonic Acid, PABA, and PEG-25 PABA.

In some instances, the UV filter is one or more of: a para-aminobenzoic acid derivative, a salicylic derivative, a cinnamic derivative, a benzophenone or an aminobenzophenone, an anthranillic derivative, a β,β-diphenylacrylate derivative, a benzylidenecamphor derivative, a phenylbenzimidazole derivative, a benzotriazole derivative, a triazine derivative, a bisresorcinyl triazine, an imidazoline derivative, a benzalmalonate derivative, a 4,4-diarylbutadiene derivative, a benzoxazole derivative, a merocyanine, malonitrile or a malonate diphenyl butadiene derivative, a chalcone, or a mixture thereof.

Suitable UV filters can include broad-spectrum UV filters that protect against both UVA and UVB radiation, or UV filters that protect against UVA or UVB radiation. In some instances, the one or more UV filters may be methylene bis-benzotriazolyl tetramethylphenol, diethylamino hydroxybenzoyl hexyl benzoate, coated or uncoated zinc oxide, ethylhexyl methoxycinnamate, isoamyl methoxycinnamate, homosalate ethyl hexyl salicilate, octocrylene, polysilicone-15, butyl methoxydibenzoylmethane, menthyl anthranilate, and ethylhexyl dimethyl PABA.

The total amount of the lipophilic active agent(s) may vary. Nonetheless, in some instances, the total amount of lipophilic active agent(s) is about 0.01 to about 10 wt. %, based on the total weight of the nanoemulsion. Similarly, in some cases, the total amount of lipophilic active agent(s) is about 0.01 to about 6 wt. %, about 0.01 to about 5 wt. %, about 0.01 to about 3 wt. %, about 0.1 to about 10 wt. %, about 0.1 to about 6 wt. %, about 0.1 to about 5 wt. %, or about 0.1 to about 3 wt. %, based on the total weight of the nanoemulsion.

Hydrophilic Active Agents

The term “hydrophilic active ingredient” (or “hydrophilic active agent”) refers to an active agent that is capable of being dissolved in water, in particular the aqueous phase of the nanoemulsion.

Non-limiting examples of useful hydrophilic active ingredients include antioxidants (e.g., vitamin C, etc.), glycerin, caffeine, botanical extracts, etc.

The total amount of the hydrophilic active agent(s) may vary. Nonetheless, in some instances, the total amount of lipophilic active agent(s) is about 0.01 to about 10 wt. %, based on the total weight of the nanoemulsion. Similarly, in some cases, the total amount of hydrophilic active agent(s) is about 0.01 to about 6 wt. %, about 0.01 to about 5 wt. %, about 0.01 to about 3 wt. %, about 0.1 to about 10 wt. %, about 0.1 to about 6 wt. %, about 0.1 to about 5 wt. %, or about 0.1 to about 3 wt. %, based on the total weight of the nanoemulsion.

Methods of Making

The instant disclosure relates to methods for making the thermoresponsive oil-in-water nanoemulsions described herein. For instance, methods for making the nanoemulsions include:
i. combining one or more oils and one or more surfactants, and optionally, one or more nonionic co-surfactants;
ii. adding water to the combination;
iii. forming a nanoemulsion and optionally adding one or more nonionic co-surfactants; and
iv. adding the one or more amphiphilic triblock copolymers to the nanoemulsion.

The entire process can be carried out at surprisingly low temperatures, for example, at temperature of about 5° C. to about 40° C. Water is typically added to the combination of oil(s), surfactant(s), and optional nonionic co-surfactant(s), and mixed to form a nanoemulsion. For example, the mixing can be carried out with magnetic stirring. The mixing is sufficiently vigorous to form a nanoemulsion. The mixing may be carried for about 1 minute to about 24 hours. After the water, the oil(s), the surfactant(s), and the optional nonionic co-surfactant(s) are sufficiently mixed such that a nanoemulsion is formed, (additional) nonionic co-surfactant(s) may optionally be added. The point at which the nonionic co-surfactant(s) is added can influence the size of the nanoemulsion droplets. After the water, oil(s), surfactant(s), and nonionic co-surfactant(s) have been combined and a nanoemulsion formed, the one or more amphiphilic triblock copolymers are added. The amphiphilic triblock copolymers are typically added in the form of a solution (e.g., an aqueous solution). After addition of the amphiphilic triblock copolymers, the nanoemulsion is thoroughly mixed. After thorough mixing, the nanoemulsion may optionally be maintained for a period of time to allow for the removal of air bubbles. For example, the thoroughly mixed nanoemulsion may be maintained at a temperature of about 0° C. to about 30° C. for about 1 hour to about 24 hours.

To prepare thermoresponsive nanoemulsions with different oil droplet sizes, the co-surfactant can be added at different times throughout the method. For example, at least part of the co-surfactant(s) can be added initially when combining the surfactant(s) and the oil(s). Also, (or alternatively) at least part of the co-surfactant(s) can be added after nanoemulsion formation. This tends to result in nanoemulsions having larger droplet sizes than if all of the nonionic co-surfactant(s) is added initially. Even larger nanoemulsion droplets can be formed if all of the nonionic co-surfactant(s) is added after initial formation of the nanoemulsion. This phenomenon is shown in Example 1.

The method of making the nanoemulsions is a low-energy phase inversion method, also known as an emulsion phase inversion. This process involves the addition of water into a stirred dispersed phase (oil, surfactant, and optional nonionic co-surfactant) at room temperature. The instant methods do not require energy-intensive emulsification processes such as high-pressure homogenization and ultrasonication. Instead, the low-energy methods only require simple mixing using, for example, a magnetic stirrer.

### EMBODIMENTS

In certain embodiments, the thermoresponsive oil-in-water nanoemulsion of the instant disclosure include:
(a) about 1 to about 15 wt. %, preferably about 1 to about 12 wt. %, more preferably about 2 to about 10 wt. % of one or more amphiphilic triblock copolymers, for example, one or more poloxamers;
(b) about 10 to about 50 wt. %, preferably about 10 to about 40 wt. %, more preferably about 15 to about 35 wt. % of one or more oils, for example, one or more ester oils, in the form of nanoemulsion droplets having an average size of about 20 nm to about 500 nm, preferably about 20 nm to about 400 nm, more preferably about 30 nm to about 200 nm;
(c) about 1 to about 25 wt. %, preferably about 2 to about 20 wt. %, more preferably about 5 to about 20 wt. % of one or more surfactants, preferably, one or more nonionic surfactants;
(d). about 0.1 to about 10 wt. %, preferably about 0.5 to about 8 wt. %, more preferably about 1 to about 7 wt. % of one or more nonionic co-surfactants, for example, one or more polyethylene glycols;
(e) about 25 to about 75 wt. %, preferably about 30 to about 70 wt. %, more preferably about 35 to about 65 wt. % of water;
(f) optionally, one or more lipophilic active ingredient; and
(g) optionally, one or more hydrophilic active ingredient;

wherein the composition undergoes a sol-to-gel transition at a temperature of about 20° C. to about 45° C., and all percentages by weight are based on the total weight of the nanoemulsion. In some instances, the sol-to-gel transition may occur at a temperature of about 25° C. to about 45° C., about 30° C. to about 45° C., about 35° C. to about 45° C., about 20° C. to about 40° C., about 25° C. to about 40° C., about 30° C. to about 40° C., about 20° C. to about 35° C., or about 25° C. to about 35° C.

The one or more amphiphilic triblock copolymers preferably includes one or more poloxamers, for example, one or more poloxamers selected from poloxamer 403 (P123), poloxamer 407 (F127), poloxamer 402 (L122), poloxamer 181 (L61), poloxamer 401 (L121), poloxamer 185 (P65), poloxamer 188 (F68) and poloxamer 338 (F108), or a mixture thereof. In some instances, a combination of poloxamer 407 (F127) and poloxamer 188 (F68) are preferred.

The one or more ester oils may be selected from diisobutyl adipate, 2-hexyldecyl adipate, di-2-heptylundecyl adipate, monoisostearic acid N-alkyl glycol, isocetyl isostearate, trimethylolpropane triisostearate, ethylene glycol di-2-ethyl hexanoate, cetyl 2-ethylhexanoate, trimethylolpropane tri-2-ethylhexanoate, pentaerythritol tetra-2-ethylhexanoate, cetyl octanoate, octyldodecyl gum ester, oleyl oleate, octyldodecyl oleate, decyl oleate, neopentyl glycol dicaprate, triethyl citrate, 2-ethylhexyl succinate, isocetyl stearate, butyl stearate, diisopropyl sebacate, di-2-ethylhexyl sebacate, cetyl lactate, myristyl lactate, isopropyl palmitate, 2-ethylhexyl palmitate, 2-hexyldecyl palmitate, 2-heptylundecyl palmitate, cholesteryl 12-hydroxystearate, dipentaerythritol fatty acid ester, isopropyl myristate, octyldodecyl myristate, 2-hexyldecyl myristate, myristyl myristate, hexyldecyl dimethyloctanoate, ethyl laurate, hexyl laurate, diisostearyl malate, dicaprylyl carbonate, and mixtures thereof.

In some instances, the one or more esters oils are preferably selected from octyldodecyl myristate, isostearyl palmitate, hexyldecyl isostearate, oleyl oleate, isocetyl myristate, stearyl stearate, decyl oleate, ethylhexyl stearate, cetyl caprate, octyl palmitate, cetyl 2-ethylhexanoate, isopropyl isostearate, hexyl laurate, isopropyl palmitate, isopropyl linoleate, and isopropyl myristate.

In further embodiments, the thermoresponsive oil-in-water nanoemulsions of the instant disclosure include:
(a) about 1 to about 15 wt. %, preferably about 1 to about 12 wt. %, more preferably about 2 to about 10 wt. % of one or more (preferably two or more) poloxamers selected from poloxamer 403 (P123), poloxamer 407 (F127), poloxamer 402 (L122), poloxamer 181 (L61), poloxamer 401 (L121), poloxamer 185 (P65), poloxamer 188 (F68) and poloxamer 338 (F108), or a mixture thereof, wherein a combination of poloxamer 407 (F127) and poloxamer 188 (F68) are preferred;
(b) about 10 to about 50 wt. %, preferably about 10 to about 40 wt. %, more preferably about 15 to about 35 wt. % of one or more ester oils selected from octyldodecyl myristate, isostearyl palmitate, hexyldecyl isostearate, oleyl oleate, isocetyl myristate, stearyl stearate, decyl oleate, ethylhexyl stearate, cetyl caprate, octyl palmitate, cetyl 2-ethylhexanoate, isopropyl isostearate, hexyl laurate, isopropyl palmitate, isopropyl linoleate, and isopropyl myristate, wherein the one or more ester oils are in the form of nanoemulsion droplets having an average size of about 20 nm to about 500 nm, preferably about 20 nm to about 400 nm, more preferably about 30 nm to about 200 nm;
(c) about 1 to about 25 wt. %, preferably about 2 to about 20 wt. %, more preferably about 5 to about 20 wt. % of one or more nonionic surfactants selected from polyol esters, glycerol ethers, oxyethylenated ethers, oxypropylenated ethers, and ethylene glycol polymers, preferably one or more nonionic surfactants selected from sorbitan fatty esters (sorbitan oleate), ethoxylated sorbitan fatty esters (polysorbate-80), and a mixture thereof;
(d). about 0.1 to about 10 wt. %, preferably about 0.5 to about 8 wt. %, more preferably about 1 to about 7 wt. % of one or more nonionic co-surfactants selected from polyethylene glycols having a molecular weight of about 100 to about 1000;
(e) about 25 to about 75 wt. %, preferably about 30 to about 70 wt. %, more preferably about 35 to about 65 wt. % of water;
(f) optionally, one or more lipophilic active ingredient; and
(g) optionally, one or more hydrophilic active ingredient;

wherein the composition undergoes a sol-to-gel transition at a temperature of about 20° C. to about 45° C., and all percentages by weight are based on the total weight of the nanoemulsion. In some instances, the sol-to-gel transition may occur at a temperature of about 25° C. to about 45° C., about 30° C. to about 45° C., about 35° C. to about 45° C., about 20° C. to about 40° C., about 25° C. to about 40° C., about 30° C. to about 40° C., about 20° C. to about 35° C., or about 25° C. to about 35° C.

In yet further embodiments, the thermoresponsive oil-in-water nanoemulsions of the instant disclosure include:
(a) about 1 to about 15 wt. %, preferably about 1 to about 12 wt. %, more preferably about 2 to about 10 wt. % of poloxamer 407 (F127) and poloxamer 188 (F68);
(b) about 10 to about 50 wt. %, preferably about 10 to about 40 wt. %, more preferably about 15 to about 35 wt. % of one or more ester oils selected from octyldodecyl myristate, isostearyl palmitate, hexyldecyl isostearate, oleyl oleate, isocetyl myristate, stearyl stearate, decyl oleate, ethylhexyl stearate, cetyl caprate, octyl palmitate, cetyl 2-ethylhexanoate, isopropyl isostearate, hexyl laurate, isopropyl palmitate, isopropyl linoleate, and isopropyl myristate, preferably at least isopropyl myristate, wherein the one or more ester oils are in the form of nanoemulsion droplets having an average size of about 20 nm to about 500 nm, preferably about 20 nm to about 400 nm, more preferably about 30 nm to about 200 nm;
(c) about 1 to about 25 wt. %, preferably about 2 to about 20 wt. %, more preferably about 5 to about 20 wt. % of one or more sorbitan fatty esters (e.g., sorbitan oleate) and one or more ethoxylated sorbitan fatty esters (e.g., polysorbate-80), and a mixture thereof;
(d). about 0.1 to about 10 wt. %, preferably about 0.5 to about 8 wt. %, more preferably about 1 to about 7 wt. % of one or more nonionic co-surfactants selected from polyethylene glycols having a molecular weight of about 100 to about 1000;
(e) about 25 to about 75 wt. %, preferably about 30 to about 70 wt. %, more preferably about 35 to about 65 wt. % of water;
(f) optionally, one or more lipophilic active ingredient; and
(g) optionally, one or more hydrophilic active ingredient;

wherein the composition undergoes a sol-to-gel transition at a temperature of about 20° C. to about 45° C., and all percentages by weight are based on the total weight of the nanoemulsion. In some instances, the sol-to-gel transition may occur at a temperature of about 25° C. to about 45° C., about 30° C. to about 45° C., about 35° C. to about 45° C., about 20° C. to about 40° C., about 25° C. to about 40° C., about 30° C. to about 40° C., about 20° C. to about 35° C., or about 25° C. to about 35° C.

Implementation of the present disclosure is provided by way of the following examples. The examples serve to illustrate the technology without being limiting in nature.

### Example 1

### Inventive Compositions

| | | A | B | C |
|---|---|---|---|---|
| Component | INCI US | (wt. %) | (wt. %) | (wt. %) |
| Nanoemulsion Droplets Size | | 53 nm | 72 nm | 115 nm |
| Amphiliphilic | Poloxamer 407 | 4 | 4 | 4 |
| Triblock | (F-127) | | | |
| Copolymers | Poloxamer 188 (F-68) | 0.7 | 0.7 | 0.7 |
| Oil | Isopropyl Myristate | 20 | 20 | 20 |
| Nonionic Surfactants | Polysorbate-80 | 16 | 16 | 16 |
| HLB = 13 | (Tween-80) | | | |
| | Sorbitan Oleate | 4 | 4 | 4 |
| | (Span-80) | | | |
| Co-Surfactant | Poly(ethylene) glycol | 5 | 5 | 5 |
| | (PEG 400) | | | |
| Water | Water | 50.3 | 50.3 | 50.3 |

Deionized (DI) water was added dropwise into a magnetically stirred mixture of oil, surfactant, and cosurfactant. Subsequent to nanoemulsion formation, a poloxamer solution (with polymer concentration of 23.3%, kept at 4° C.), was added. To thoroughly mix the final solution, it was vortexed for additional 5 seconds and kept at 4° C. overnight (to remove air bubbles). The thermoresponsive nanoemulsion (Composition A) had a droplet size of 53±2 nm with polydispersity index (PDI) of 0.12, as measured using dynamic light scattering.

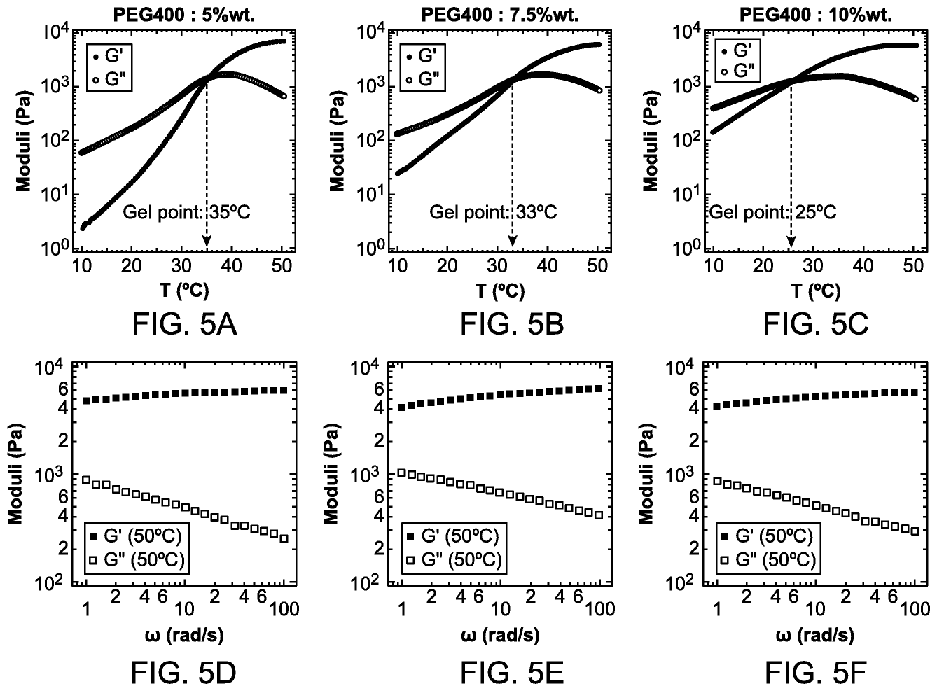

To prepare thermoresponsive nanoemulsions with different oil droplet sizes (53 nm, 72 nm, and 115 nm), the co-surfactant (PEG 400, total of 5% wt.) was added during and/or after nanoemulsion preparation. A nanoemulsion with oil droplet size of 72 nm (Composition B) was prepared by adding 2.5% wt. PEG 400 during and then 2.5% wt. after nanoemulsion formation. Similarly, adding 5% wt. PEG 400 after nanoemulsion formation resulted in droplet size of 115 nm (Composition C). In addition, to obtain nanoemulsions with different concentrations of co-surfactant (e.g., PEG 400) while having a similar oil droplet (see FIG. 5), 5% cosurfactant was used during nanoemulsion formation and an additional amount of co-surfactant was incorporated after forming the nanoemulsions.

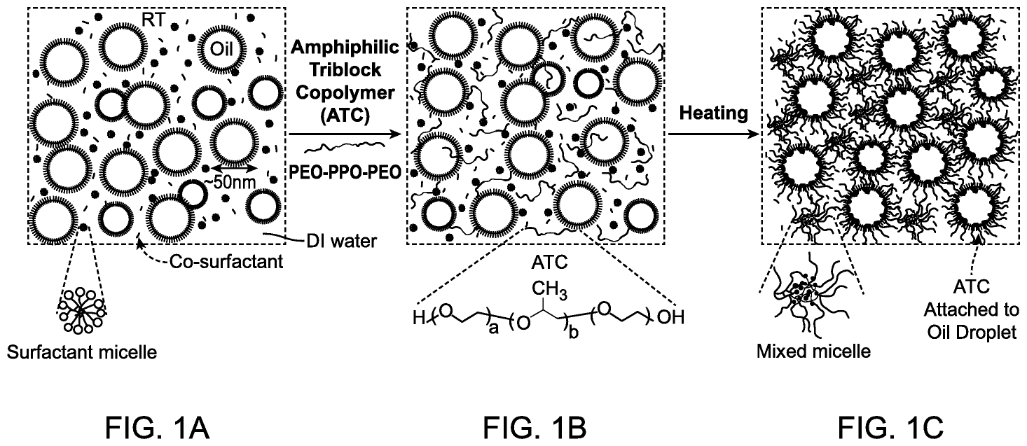

The oil-in-water nanoemulsions were produced using a low-energy phase inversion composition method, also known as emulsion phase inversion (FIG. 1a). This process involves the addition of water into a stirred dispersed phase (oil, surfactant, and co-surfactant) at room temperature. The optimal surfactant HLB (hydrophilic-lipophilic balance) was considered to minimize droplet size. Poly(ethylene glycol), PEG 400 (5% wt.), was used as a co-surfactant to further reduce the oil droplet size. In contrast to energy-intensive emulsification processes such as high pressure homogenization and ultrasonication, the low-energy method only requires simple mixing using, for example, mixing using a magnetic stirrer. After nanoemulsion preparation (with droplet size of 53 nm, PDI of 0.12), the poloxamers were dispersed into the suspension to render it thermally responsive (FIG. 1b). A mixture of poloxamers with a final concentration of 4.7% wt. was used to tune the gelation temperature. The resulting thermoresponsive nanoemulsions were very durable and remained stable for a period of at least six months.

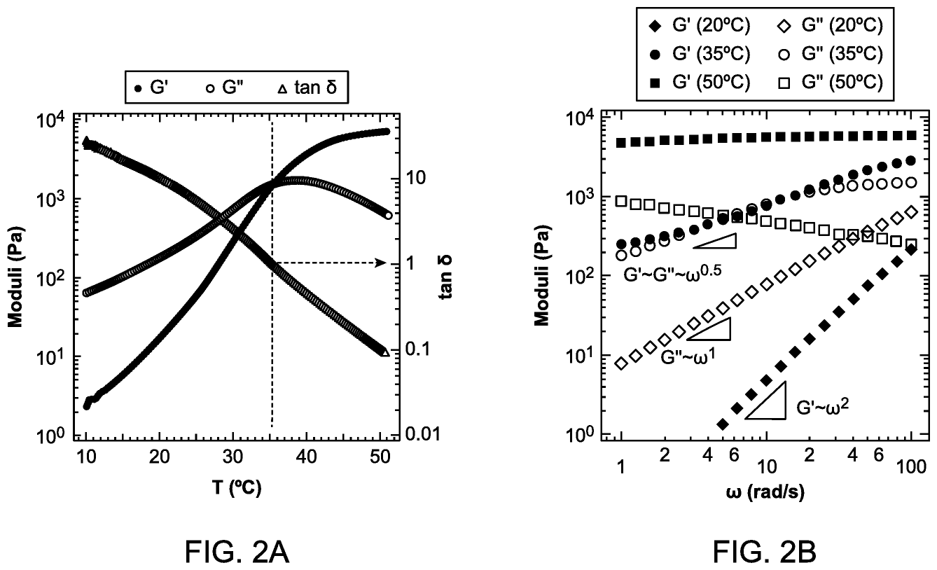

The sol-to-gel transition of the thermoresponsive nanoemulsion is displayed in FIG. 2. Evolution of gel formation as a function of temperature was captured in small amplitude oscillatory shear rheology and vial inversion tests. Temperature sweep measurements at a fixed frequency (FIG. 2a) show that both elastic (G′) and loss (G″) moduli increase as a function of temperature starting from 10° C. and crossover each other at a critical gel point (T_{gel}=35° C.). Above the gel point, G′ exceeds G″ and at 50° C. the elastic modulus eventually becomes about one order of magnitude higher than the loss modulus. Frequency sweep tests were conducted at temperatures corresponding to the liquid state, critical gel point, and gel state, FIG. 2b. At low temperature (20° C.), where G″>>G′, G′(ω)˜ω² and G″(ω)˜ω¹ which indicate liquid-like behavior. However, at the critical gel point, the moduli display identical power-law scaling with frequency for over two decades of frequency (G′(ω)˜G″(ω)˜ωⁿ, here with n=0.5) which is consistent with the Winter-Chambon criteria for gelation. The same power law dependency of moduli with n=0.5 is also shown in tan δ (tan δ=G″(ω)/G′(ω))), FIG. 2a. At the G′-G″ crossover, tan δ is equal to 1 which corresponds to the power exponent of 0.5 (i.e. tan nπ/2≈1, n=0.5). The frequency dependence of the shear moduli above the gel point continues to decrease for both the elastic and loss components. In the gel state (50° C., FIG. 2b), G′ becomes relatively frequency independent (G′(ω)˜ω⁰) and the power exponent of G″ is a negative number, similar to a Maxwell model for non-Newtonian viscoelastic materials This high temperature elastic modulus is stronger than reported for other liquid-liquid emulsion systems. In the liquid state the nanoemulsions are able to flow, whereas at gel state the self-assembled nanoemulsions can hold their own weight. Turbidity of the nanoemulsion solution remained visually unchanged upon gelation—both liquid and gel states are translucent.

Control experiments were performed to verify the essential role of the nanoemulsion droplets in the thermogelling behavior. Poloxamers are amphiphilic triblock copolymers of poly(ethylene oxide)ₐ-poly(propylene oxide)_{b}-poly(ethylene oxide)ₐ (PEO-PPO-PEO). As temperature increases, midblock segments become more hydrophobic, while PEO segments are fully hydrated over a wide range of temperatures. The temperature-dependent solubility of PPO in water promotes micelle formation. At high enough concentration, these spherical micelles construct a packed microstructure which immobilizes the aqueous phase through gelation. Temperature ramp measurements of the mixed poloxamers indicated that the minimum concentration of poloxamers alone needed for sol-to-gel transition to occur is 13% and at 15%, only a weak gel can be obtained. Additionally, dynamic rheological experiments were conducted on nanoemulsions without the presence of the oil and no gel formation was observed over the temperature range 10° C. to 60° C.

These control experiments show that the presence and function of the oil droplets is critical in obtaining the described thermoresponsive nanoemulsions. While not wishing to be bound by any particular theory, it is believed that the thermally induced gelling occurs via a synergistic interaction between the poloxamers and the nanoemulsion droplets (FIG. 1c). There are multiple hydrophobic sites that may affect the self-assembly of poloxamers as temperature rises. These include nanoemulsion oil droplets as well as the hydrophobic core of surfactant micelles. As the temperature increases, PPO segments of poloxamers absorb to the oil interface as they become more hydrophobic. The adsorbed layer of poloxamers onto the oil interface alters the effective volume fraction (ϕ_{eff}) of the oil droplets. The effective volume fraction of the poloxamer-coated “hairy” droplets can be estimated using the relationship derived for electrostatically stabilized suspensions: ϕ_{eff}=ϕ(δ/r)³, where δ is the droplet radius with adsorbed layer of poloxamer, r the is initial nanoemulsion radius, and ϕ is the true volume fraction. Assuming an adsorbed layer thickness of ≈5.5 nm, the effective volume fraction of the oil phase can be increased from 0.24 to 0.44 upon adsorption of the poloxamer. While this is a significant increase in the effective volume fraction, it is still below the random close-packed limit for monodisperse hard spheres (ϕ=0.64). Excess poloxamer can also form mixed micelles in the aqueous phase which increases the micelle concentration in the solution (FIG. 1c). Akin to the jamming-induced gelation in pure poloxamer systems, it is believed that the packing of the mixed-micelles with the hairy-nanodroplets leads to the observed thermos-responsiveness. The fact that there is no gel transition in the absence of oil droplets suggests that the adsorption of poloxamer onto the droplet interface is a key step which gives rise to the thermogelling behavior.

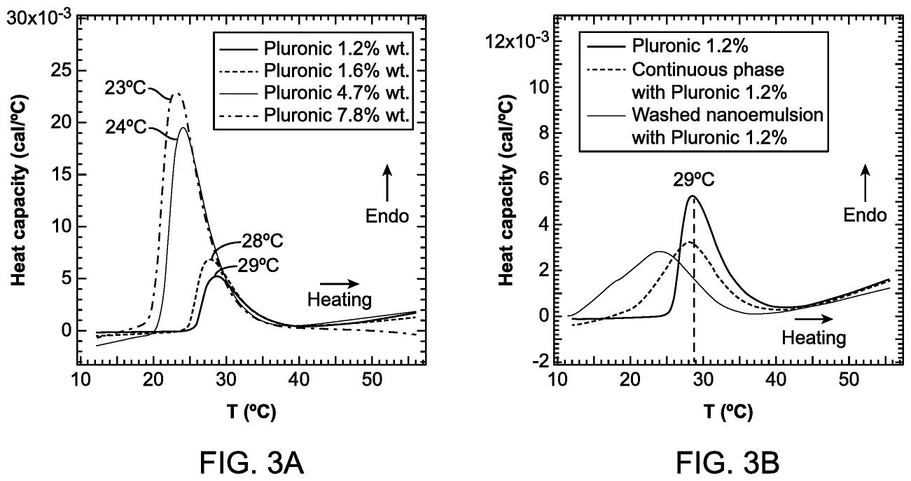

To corroborate the hypothesis regarding the underlying thermogelling mechanism, microcalorimetry measurements were carried out (FIG. 3). To find the critical micellization temperature of the poloxamer solution, micro-DSC measurements were conducted on different concentrations (FIG. 3a). The thermograms of the poloxamer solutions give rise to an endothermic peak as a result of heating. As temperature increases, PPO segments become dehydrated and less polar which eventually aggregate to form micelles resulting in an endothermic peak in the micro-DSC. The micellization process depends on the concentration and chain length of the poloxamers. For example, peak temperature decreases from 29° C. to 23° C. as the poloxamer concentration increases from 1.2% to 7.8%. To evaluate the adsorption of the midblock (PPO) in the poloxamers onto hydrophobic oil interfaces during ramping temperature experiments, the thermogelling nanoemulsion was diluted to decrease the viscosity of the solution sufficiently to load the samples for micro-DSC measurements.

Nanoemulsions were centrifuged (using centrifugal filters) to filter the oil droplets from the continuous phase and were separately analyzed in DSC experiments. FIG. 3b displays DSC traces for the neat poloxamer solution, the subnatant solution (continuous phase), and the washed nanoemulsion (oil) with a similar poloxamer concentration. Interestingly, the endothermic transition peak broadens in the presence of oil droplets and the onset of the peak significantly shifts 13° C. to a lower temperature. As temperature increases in the presence of the oil droplets, hydrophobic segments of the poloxamers (PPO groups) are adsorbed onto the oil droplets and results in an endothermic peak. This implies that poloxamers and nanoemulsion droplets interact strongly with each other. In addition, increased micelle formation occurs in the continuous phase. The endothermic transition peak (FIG. 3b) was centered at 29° C., similar to the neat poloxamer solution; however, the onset of the endothermic peak shifted 5° C. to a lower temperature. This early onset is likely due to the mixed micelle formation (as shown in FIG. 1c) between poloxamer and free surfactant molecules (polysorbate-80 and sorbitan oleate) present in the aqueous phase. The DSC data suggests that the endothermic adsorption process of the poloxamers onto the oil droplet interface is more favorable and dominant as compared with micellization of the poloxamers themselves.

### Example 2

### Influence of Nanoemulsion Droplet Size

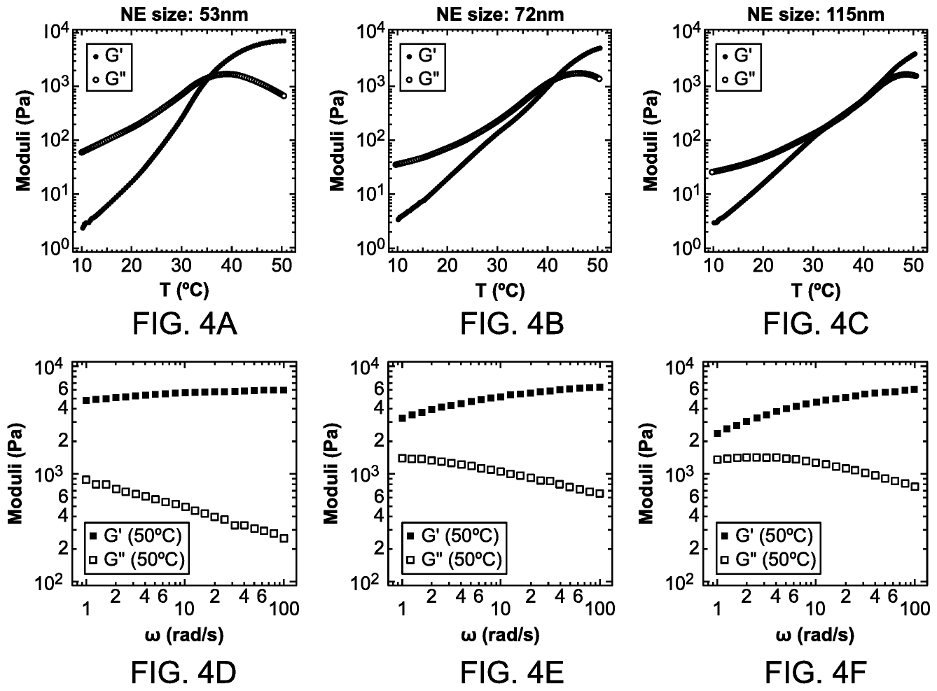

Nanoemulsions with varied droplet diameters were prepared to examine the effect of droplet size on the thermogelling behavior. All compositions were identical, except for the oil droplet sizes. FIG. 4 displays the shear moduli as a function of temperature as well as frequency dependency of the moduli at 50° C. for three different droplet sizes (53 nm, 72 nm, and 115 nm). All three nanoemulsions show thermogelling, though with some qualitative changes in rheological behavior and shifts in gelation temperature. For the nanoemulsion with the largest droplet size (115 nm), the G′-G″ crossover spans from 30° C. to more than 40° C. At 50° C., the storage modulus becomes more frequency dependent as droplet size increases. At a fixed dispersed phase volume fraction, increasing the nanoemulsion size from 53 nm to 115 nm reduces the oil surface area by approximately a factor of 2. Additionally, increasing the droplet size (holding the true volume fraction constant) decreases the effective volume fraction for poloxamer-coated droplets from ϕ_{eff}≈0.44 to 0.33 when increasing the droplets size from 53 to 115 nm. Decreasing the effective volume fraction of the oil droplet likely results in the formation of a gel with a less jammed structure at elevated temperatures and decrease in elasticity.

### Example 3

### Influence of Co-Surfactant

It was discovered that the rheological behavior of the nanoemulsions can be modulated by other factors in addition to nanoemulsion droplet size. To test the effects of a co-surfactant (PEG 400) on thermogelling behavior, the concentration of the co-surfactant was varied from 5 wt. % to 10 wt. %. The viscoelastic moduli as a function of temperature are shown in FIG. 5. Interestingly, increasing the concentration of PEG 400 lowers the gelation temperature. For example, the gel point reduces by 10° C., when PEG 400 is doubled from 5% to 10%. In addition, both the storage and the loss modulus have higher values in the liquid state with an increase of PEG 400 concentration. However, all three nanoemulsions have similar shear moduli in the gel state (50° C.). The frequency sweep data captured at the gel state also indicate a similar frequency dependence of shear moduli for these thermogelling nanoemulsions. Having higher values of viscoelastic moduli in the liquid state and lower gelation temperatures with increasing concentration of PEG 400, indicate possible H-bonding between hydroxyl groups and ether groups among PEG 400 molecules or between PEG 400 and polyethylene groups in the poloxamers. Similar results showed that the presence of short polyethylene glycol lowers the critical micellization temperature of poloxamer aqueous solutions.

### Example 4

### Rheological Properties of Nanoemulsions

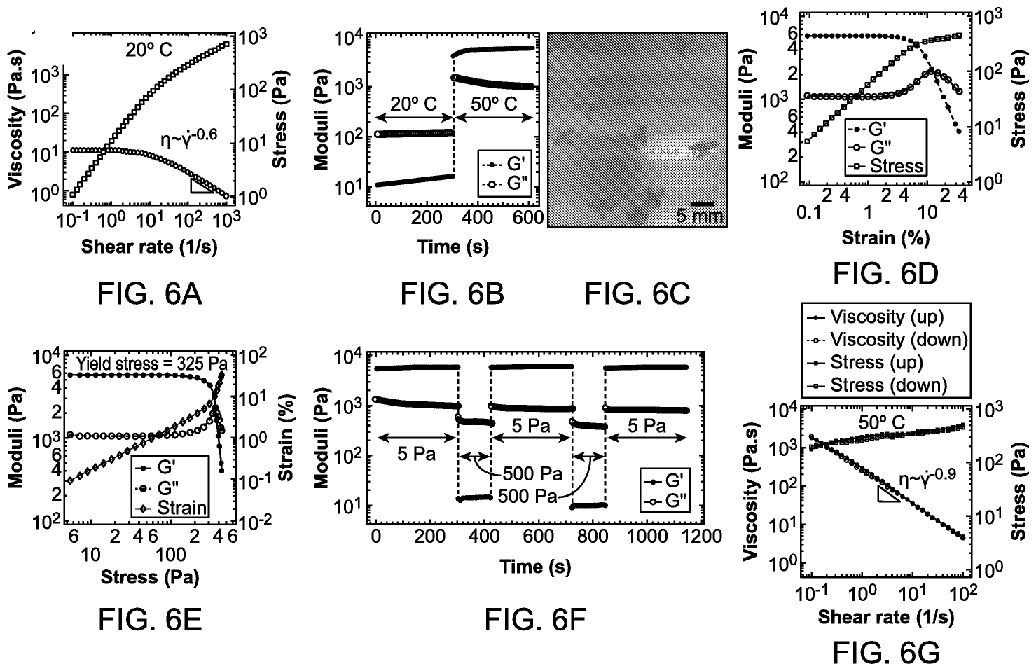

For a thermogelling material to be used as a topical formulation, certain rheological properties are desirable both below and above the gelation temperature. At ambient temperature, flow behavior is similar to Newtonian fluids at shear rates lower than 1 s⁻¹; that is, shear stress linearly increases against shear rate and corresponding viscosity is independent of shear rate (a plateau in the viscosity at low shear rates) (FIG. 6a). However, at higher shear rates, the nanoemulsions become shear-thinning response with a power-law dependence on shear rate, η˜\dot{γ}^{m} with m=−0.6. The shear thinning properties at ambient temperature allows for easy injection of the material. After the nanoemulsion is ejected on a target surface at an elevated temperature, it should undergo a rapid phase transition in order to preserve the structure. To test the material response, qualitative and quantitative temperature-jump experiments were performed. FIG. 6b displays shear moduli in a temperature jump test from 20° C. to 50° C. Intriguingly, the thermogelling nanoemulsion responds very quickly to a temperature jump and becomes a gel almost instantaneously. In order to demonstrate qualitatively the gelation rate, the room temperature thermogelling nanoemulsion was dripped through a flat-tip 15-gauge needle into hot water at 50° C. (FIG. 6c). For better observation of the process, the oil phase was also loaded with the Nile red (0.05 mg/mL). The drops of the thermogelling nanoemulsion liquid-like suspension become a gel as they enter the hot water and form mushroom-like objects. These immersed gel objects retain their shape and persist for several minutes before eventually dissolving into the water.

### Example 5

### Flow Behavior

Large amplitude oscillatory shear (LAOS) experiments were conducted to monitor the nonlinear flow behavior of the nanoemulsion. FIG. 6d displays shear moduli and stress as a function of applied shear strain amplitude at 50° C., where the gelled material is highly structured. The storage and loss moduli are independent of strain below a strain amplitude of 2%, indicating a linear viscoelastic regime. At higher applied strain, a starts to deviate from linearity with a gradual drop in magnitudes and above strain of 10%, a sharp decay was observed. Conversely, G″ starts to grow beyond the linear regime and passes through a maxima exceeding G′ prior to a sharp decay resulting from complete structural failure of the material. Similar LAOS results have also been reported structure for emulsion-based gels with a peak in G″ at large strain. The transition from linear to nonlinear viscoelastic regimes can also be monitored in a stress-strain curve. Shear stress linearly increases as a function of strain amplitude up to a stress 110 Pa, beyond which point stress starts to deviate from linearity. In order to better estimate the yield stress, the critical point above which the structured thermogelling nanoemulsion is disrupted and starts to flow, the shear moduli were plotted as a function of stress (FIG. 6e). Two tangent lines were plotted in the linear and the nonlinear regimes of a and the crossover point determines the yield point. The obtained a yield stress of 325 Pa at the gel state is comparable to the yield stress of pure poloxamer gels with a much higher copolymer mass fraction of 19%.

### Example 6

### Recovery of Elasticity

To test recovery of the elasticity after yielding, low to high oscillatory shear stress was applied repeatedly to the nanoemulsions. FIG. 6f displays the moduli as a function of time after two cycles of low (5 Pa for 120 s, below the yield stress) to high (500 Pa for 300 s, above the yield stress) oscillatory stress amplitudes. The application of a shear stress above the material yield stress resulted in the moduli indicative of liquid-like behavior (G″>>G′). However, the structure can recover its full elastic strength after cessation of the applied large stress. Full recovery of internal structure in the thermogelling nanoemulsion occurs nearly instantly (in less than 10 seconds). In addition, after two cycles of low to high oscillatory stress, the material returned to its initial value of elastic modulus. In order to further investigate the time-dependent rheological properties, the thixotropic features (breakdown and buildup of the internal structure) were monitored. An established protocol for characterizing the thixotropic behavior was applied in which the shear viscosity and stress was monitored under steady flow cycle of progressively increasing and decreasing shear rate (FIG. 6g). Interestingly, the initially gelled material at T=50° C. shows strong shear thinning behavior (η˜\dot{γ}^{m} with =−0.9). Additionally, the viscosity is not dependent on time, indicated by overlap in viscosity recorded from low to high shear rate, and the reverse. These results also confirm that the full recovery occurs nearly instantaneously after the material is disrupted (as shown in FIG. 6f) to prevent the material from flowing or dripping.

All publications, patent applications, and journal articles cited in this specification are herein incorporated by reference, and for any and all purposes, as if each individual publication were specifically and individually indicated to be incorporated by reference. In the event of an inconsistency between the present disclosure and any publications incorporated herein by reference, the present disclosure controls.

The terms “comprising,” “having,” and “including” are used in their open, non-limiting sense.

The terms “a” and “the” are understood to encompass the plural as well as the singular.

The compositions (e.g., the “nanoemulsions”) and methods of the present disclosure can comprise, consist of, or consist essentially of the essential elements and limitations of the disclosure described herein, as well as any additional or optional ingredients, components, or limitations described herein or otherwise useful.

All percentages, parts and ratios herein are based upon the total weight of the compositions of the present disclosure, unless otherwise indicated.

All ranges and values disclosed herein are inclusive and combinable. For examples, any value or point described herein that falls within a range described herein can serve as a minimum or maximum value to derive a sub-range, etc. Furthermore, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc.

Other than in the operating examples, or where otherwise indicated, all numbers expressing quantities of ingredients and/or reaction conditions are to be understood as being modified in all instances by the term “about,” meaning within +/−5% of the indicated number.

As used herein, the expression “at least one” is interchangeable with the expression “one or more” and thus includes individual components as well as mixtures/combinations.

The term “substantially free” or “essentially free” as used herein means that there is less than about 2% by weight of a specific material added to a composition, based on the total weight of the compositions. Nonetheless, the compositions may include less than about 1 wt. %, less than about 0.5 wt. %, less than about 0.1 wt. %, less than 0.01 wt. %, or none of the specified material.

The term “active material” as used herein with respect to the percent amount of an ingredient or raw material, refers to 100% activity of the ingredient or raw material.

“Cosmetically acceptable” means that the item in question is compatible with a keratinous substrate such as skin and hair. For example, a “cosmetically acceptable carrier” means a carrier that is compatible with a keratinous substrate such as skin and hair. The nanoemulsions described herein are preferably cosmetically acceptable.

Throughout the disclosure, the term “a mixture thereof” may be used following a list of elements as shown in the following example where letters A-F represent the elements: “one or more elements selected from the group consisting of A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counter-ion. This list of counter-ions, however, is non-limiting.

The phrase “stable emulsion” (or “stable nanoemulsion”) refers to a composition (i.e., emulsion/nanoemulsion) that does not undergo phase separation up to a temperature of 45° C. for at least two weeks. In some instances, the nanoemulsions of the instant disclosure remain stable for at least six months.

The expression “inclusive” for a range of concentrations means that the limits of the range are included in the defined interval.

“Volatile”, as used herein, means having a flash point of less than about 100° C.

“Non-volatile”, as used herein, means having a flash point of greater than about 100° C.

The term “polymers,” as defined herein, include homopolymers and copolymers formed from at least two different types of monomers.

The term “INCI” is an abbreviation of International Nomenclature of Cosmetic Ingredients, which is a system of names provided by the International Nomenclature Committee of the Personal Care Products Council to describe personal care ingredients.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc.

All components and elements positively set forth in this disclosure can be negatively excluded from the claims. In other words, the compositions (nanoemulsions) of the instant disclosure can be free or essentially free of all components and elements positively recited throughout the instant disclosure.

Some of the various categories of components identified may overlap. In such cases where overlap may exist and the composition includes both components (or the composition includes more than two components that overlap), an overlapping compound does not represent more than one component. For example, a fatty acid may be characterized as both a nonionic surfactant and a fatty compound. If a particular composition includes both a nonionic surfactant and a fatty compound, a single fatty acid will serve as only the nonionic surfactant or as only the fatty compound (the single fatty acid does not serve as both the nonionic surfactant and the fatty compound).

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A thermoresponsive oil-in-water nanoemulsion comprising:
(a) about 1 to about 15 wt. % of one or more amphiphilic triblock copolymers;
(b) about 10 to about 50 wt. % of one or more oils in the form of nanoemulsion droplets having an average size of about 20 nm to about 500 nm;
(c) about 1 to about 25 wt. % of one or more surfactants;
(d). about 0.1 to about 10 wt. % of one or more nonionic co-surfactants; and
(e) about 25 to about 75 wt. % of water;
wherein the composition undergoes a sol-to-gel transition at a temperature of about 20° C. to about 45° C., and all percentages by weight are based on the total weight of the nanoemulsion.

2. The nanoemulsion of claim 1 comprising two or more amphiphilic triblock copolymers.

3. The nanoemulsion of claim 1, wherein the amphiphilic triblock copolymer(s) are selected from poloxamers.

4. The nanoemulsion of claim 2 comprising poloxamer 407 and poloxamer 188.

5. The nanoemulsion of claim 1 comprising nanoemulsion droplets with an average size of about 20 nm to about 400 nm.

6. The nanoemulsion of claim 1, wherein the one or more oils are selected from ester oils.

7. The nanoemulsion of claim 6, wherein the one or more ester oils are selected from of diisobutyl adipate, 2-hexyldecyl adipate, di-2-heptylundecyl adipate, monoisostearic acid N-alkyl glycol, isocetyl isostearate, trimethylolpropane triisostearate, ethylene glycol di-2-ethylhexanoate, cetyl 2-ethylhexanoate, trimethylolpropane tri-2-ethylhexanoate, pentaerythritol tetra-2-ethylhexanoate, cetyl octanoate, octyldodecyl gum ester, oleyl oleate, octyldodecyl oleate, decyl oleate, neopentyl glycol dicaprate, triethyl citrate, 2-ethylhexyl succinate, isocetyl stearate, butyl stearate, diisopropyl sebacate, di-2-ethylhexyl sebacate, cetyl lactate, myristyl lactate, isopropyl palmitate, 2-ethylhexyl palmitate, 2-hexyldecyl palmitate, 2-heptylundecyl palmitate, cholesteryl 12-hydroxystearate, dipentaerythritol fatty acid ester, isopropyl myristate, octyldodecyl myristate, 2-hexyldecyl myristate, myristyl myristate, hexyldecyl dimethyloctanoate, ethyl laurate, hexyl laurate, diisostearyl malate, dicaprylyl carbonate, and mixtures thereof.

8. (canceled)

9. The nanoemulsion of claim 1, wherein the one or more surfactants are selected from nonionic surfactants.

10. The nanoemulsion of claim 9, wherein the one or more nonionic surfactants are selected from polyol esters, glycerol ethers, oxyethylenated ethers, oxypropylenated ethers, and ethylene glycol co-polymers.

11. The nanoemulsion of claim 10, wherein the one or more nonionic surfactants are selected from sorbitan fatty esters (sorbitan oleate), ethoxylated sorbitan fatty esters (polysorbate-80), and a mixture thereof.

12. The nanoemulsion of claim 11 comprising sorbitan oleate and polysorbate-80.

13. The nanoemulsion of claim 1, wherein the one or more nonionic co-surfactants are selected from polyethylene glycols having a molecular weight of about 100 to about 1000.

14. (canceled)

15. The nanoemulsion of claim 1 that is essentially free of silicones.

16. The nanoemulsion of claim 1 that is translucent.

17. The nanoemulsion of claim 1, further comprising one or more lipophilic active ingredients.

18. The nanoemulsion of claim 1, further comprising one or more hydrophilic active ingredients.

19. A thermoresponsive oil-in-water nanoemulsion comprising:
(a) about 1 to about 15 wt. % of two or more poloxamers;
(b) about 10 to about 40 wt. % of one or more ester oils in the form of nanoemulsion droplets having an average size of about 20 nm to about 300 nm;
(c) about 1 to about 25 wt. % of one or more nonionic surfactants selected from sorbitan fatty esters, ethoxylated sorbitan fatty esters, and a mixture thereof;
(d) about 1 to about 15 wt. % of one or more nonionic co-surfactants selected from polyethylenge glycols; and
(e) about 25 to ab out 75 wt. % of water.
wherein the composition undergoes a sol-to-gel transition at a temperature of about 25° C. to about 45° C., and all percentages by weight are based on the total weight of the nanoemulsion.

20. The nanoemulsion of claim 19 comprising:
(a) about 1 to about 15 wt. % of poloxamer 407 and poloxamer 188;
(b). about 10 to about 40 wt. % of isopropyl myristante in the form of nanoemulsion droplets having an average size of about 20 nm to about 300 nm;
(c) about 1 to about 25 wt. % of sorbitan oleate and polysorbate-80);
(d) about 1 to about 15 wt. % of one or more nonionic co-surfactants selected from polyethylenge glycols; and
(e) about 25 to ab out 75 wt. % of water.

21. A method of making the thermoresponsive oil-in-water nanoemulsion of claim 1 comprising:
i. combining the one or more oils, the one or more surfactants, and the one or more nonionic co-surfactants;
ii. adding water to the combination;
iii. forming a nanoemulsion; and
iv. adding the one or more amphiphilic triblock copolymers to the nanoemulsion.

22. (canceled)

23. A method for treating the skin and/or hair comprising applying the nanoemulsion of claim 1 to the skin and/or hair.
